SEQUENCE LISTING (21 sequences)

SEQ ID NO: 1 - DNA, 25 bp, Artificial
  Primer capable of hybridizing LAMDA DNA
gatgagttcgtgtccgtacaactgg

SEQ ID NO: 2 - DNA, 25 bp, Artificial
  Primer capable of hybridizing LAMDA DNA
gatgagttcgtgtccgtacaactga

SEQ ID NO: 3 - DNA, 25 bp, Artificial
  Primer capable of hybridizing LAMDA DNA
gatgagttcgtgtccgtacaactgt

SEQ ID NO: 4 - DNA, 25 bp, Artificial
  Primer capable of hybridizing LAMDA DNA
gatgagttcgtgtccgtacaactct

SEQ ID NO: 5 - DNA, 25 bp, Artificial
  Primer capable of hybridizing LAMBDA DNA
gatgagttcgtgtccgtacaactca

SEQ ID NO: 6 - DNA, 25 bp, Artificial
  Primer capable of hybridizing LAMBDA DNA
gatgagttcgtgtccgtacaacact

SEQ ID NO: 7 - DNA, 25 bp, Artificial
  Primer capable of hybridizing LAMBDA DNA
gatgagttcgtgtccgtacaacaca

SEQ ID NO: 8 - DNA, 25 bp, Artificial
  Primer capable of hybridizing LAMBDA DNA
gatgagttcgtgtccgtacaacacc

SEQ ID NO: 9 - DNA, 25 bp, Artificial
  Primer capable of hybridizing LAMBDA DNA
gaatcacggtatccggctgcgctga

SEQ ID NO: 10 - DNA, 520 bp, Homo sapiens
agagatgccttcccctgtagcagtcttcagcctcctctaccctacragatctggagcaacagctaggaaatatcattaattcagctcttcagagatgttatcaataaattacacatgggggctttccaaagaaatggaaattgatgggaaattatttttcaggaaaatttaaaattcaagtgagaagtaaataaagtgttgaacatcagctggggaattgaagccaacaaaccttccttcttaaccattctactgtgtcacctttgccattgaggaaaaatattcctgtgacttcttgcatttttggtatcttcataatctttagtcatcgaatcccagtggaggggacccttttacttgccctgaacatacacatgctgggccattgtgattgaagtcttctaactctgtctcagttttcactgtcgacattttcctttttctaataaaaatgtaccaaatccctggggtaaaagctagggtaaggtaaaggatagactcacatttacaagtagtgaag

SEQ ID NO: 11 - DNA, 26 bp, Artificial
  Typing primer
cagtcttcagcctcctctacccttga

SEQ ID NO: 12 - DNA, 26 bp, Artificial
  Typing primer
cagtcttcagcctcctctacccttgg

SEQ ID NO: 13 - DNA, 30 bp, Artificial
  Reverse primer
cttcactacttgtaaatgtgagtctatcct

SEQ ID NO: 14 - DNA, 28 bp, Artificial
  Typing primer
agcagtcttcagcctcctctacccttgg

SEQ ID NO: 15 - DNA, 24 bp, Artificial
  Reverse primer
attaatgatatttcctagctgttg

SEQ ID NO: 16 - DNA, 360 bp, Homo sapiens
aatggggaaccttgaagcagagaccaatgttttggtsctgaggctggttcagaaaaaggatttttaaaaaaagtatgtaatttttaaaagttctgatgattagaacacagacctcaggaaagtagcgtgaacatactgctggcgatggtagcagcttcgttggtttagcaaagtgacagaagtatctatttggagtgtttttctgaccctgacacggtatgtggaggtggatgaaagcagcgaagtttcatctgagaaccgtaagggttttcccttttcttacttgcttcccatttaaatcagtgcaagagagaatatgaatttataatgctttacttgggatgcctgtggaatatgttg

SEQ ID NO: 17 - DNA, 20 bp, Artificial
  primer
aatggggaaccttgaagcag

SEQ ID NO: 18 - DNA, 20 bp, Artificial
  Reverse primer
caacatattccacaggcatc

SEQ ID NO: 19 - DNA, 24 bp, Artificial
  Typing primer
gaagcagagaccaatgttttgcag

SEQ ID NO: 20 - DNA, 24 bp, Artificial
  Typing primer
gaagcagagaccaatgttttgcac

SEQ ID NO: 21 - DNA, 24 bp, Artificial
  Primer
agagatgccttcccctgtagcagt

(19) United States
(12) Patent Application Publication
(10) Pub. No.: US 2004/0197803 A1
(43) Pub. Date: Oct. 7, 2004

(54) Method, primer and kit for determining base type

(76) Inventors: Hidenobu Yaku, Kadoma (JP); Tomomi Suzuki, Kyoto (JP); Tetsuo Yukimasa, Nara (JP); Hiroaki Oka, Hirakata (JP)

Correspondence Address: WENDEROTH, LIND & PONACK, L.L.P., 2033 K STREET N. W., SUITE 800, WASHINGTON, DC, 20006-1021, US

(21) Appl. No.: 10/727,664

(22) Filed: Dec. 5, 2003

(30) Foreign Application Priority Data
Dec. 6, 2002 (JP) 2002-355915
Aug. 7, 2003 (JP) 2003-288707

(51) Int. Cl. C12Q 1/68

(52) U.S. Cl. 435/6

(57) ABSTRACT

A base type determination primer for determining a base type of a monobasic substituted region of a target nucleic acid, wherein the primer consists of a single-stranded nucleic acid which is capable of hybridizing to the target nucleic acid such that a 3′ terminal thereof corresponds to the substituted region of the target nucleic acid, and wherein the single-stranded nucleic acid includes: a substitution corresponding region located at the 3′ terminal and consisting of a base complementary to any one of predictable types of bases in the substituted region of the target nucleic acid; an uncomplementary region adjacent to the substitution corresponding region on the 5′ terminal side thereof and consisting of two bases uncomplementary to the target nucleic acid; and a complementary region adjacent to the uncomplementary region on the 5′ terminal side thereof and consisting of five or more bases complementary to the target nucleic acid.

### BACKGROUND OF THE INVENTION

1. Field of the Invention

The present invention relates to a method for determining the type of a target base in a nucleic acid base sequence, and also relates to a primer and kit for use in such a method.

2. Description of the Background Art

In February, 2001, Human Genome Project (HGP) and Celera Genomics, USA, published the draft sequence of the human genome, which is a major milestone, especially in the medical field. However, it will take a while before the draft sequence of the human genome is utilized in our life or medical treatment. The published draft sequence is merely resulted from rough determination of base sequences of the human genome, and it is a major issue for the future to clarify functions of individual genes in the human genome and thereby to clarify mechanisms of various diseases. Currently, most of the attention in the field is attracted towards single nucleotide polymorphisms (SNPs) in DNA.

The SNP is one of the most observed gene polymorphisms among individuals of the same species, and more specifically, the SNP corresponds to a base-pair difference (i.e., a substitution) observed at a specific site in a base sequence of genomic DNA. It is estimated that there exists one SNP in hundreds to one thousand base pairs.

The SNP serves as a polymorphism marker which is extremely useful in search of a gene related to, for example, liability to disease, responsiveness to a specific drug, and side effects therefrom. Some SNPs may influence gene expression regulation. For example, when there is a change in amino acid in a protein, which corresponds to a region including the SNP in a DNA base sequence, the function of the protein is influenced by such a change, resulting in qualitative and/or quantitative abnormalities of other gene products. In such a case, the SNP itself can be used for measuring individual differences in liability to disease, responsiveness to a specific drug, and side effects therefrom, for example.

Accordingly, it is extremely important to conduct research and study of the SNP. Currently in Japan, research and study of the SNP is actively conducted, especially with respect to the five major diseases. It is considered to take another three to five years before results of such research and study of the SNP become available in the actual medical treatment so that our own SNPs are diagnosed in clinical settings, etc. Therefore, extreme importance is placed on so-called SNP site base type determining technology (hereinafter, such technology is referred to as the “SNP typing technology”).

Although it is not a case of the SNP, there is a known example where mutation of only one base pair in the base sequence of the genomic DNA leads to a very severe disease. Therefore, it is also becoming extremely important to determine the presence or absence of such a base-pair substitution. The SNP typing technology is useful in determining the presence or absence of the base-pair substitution.

Currently, a variety of types of SNP typing techniques are under development or have already been put into practical use. One of the simplest among such SNP techniques uses a primer extension reaction. In this technique, SNP typing is conducted by determining a difference in progress of the primer extension reaction using a primer having a base sequence complementary to a base sequence adjacent to an SNP site in target nucleic acid and causing a difference in progress of the extension reaction in accordance with the type of a base at the SNP site in the target nucleic acid (hereinafter, such a primer is referred to as a “typing primer”).

In many cases, the difference in progress of the primer extension reaction of a typing primer as described above is analyzed using a reaction which amplifies a target base sequence, e.g., a polymerase chain reaction (PCR), a nucleic acid sequence-based amplification (NASBA), a ligase chain reaction (LCR), a strand displacement amplification (SDA), a rolling circle replication (RCR), a loop-mediated isothermal amplification (LAMP), or a transcription mediated amplification (TMA). After the reaction, the degree of amplification of the target base sequence is analyzed by an electrophoresis method or the like, thereby typing the SNP site. Regarding exemplary cases where the target base sequence is amplified using a PCR, and thereafter a difference in progress of the primer extension reaction is analyzed by electrophoresis, detailed description is provided below with reference to FIGS. 1A through 3.

Firstly, in the step shown in FIG. 1A, a sample solution containing a target double-stranded DNA 1 having an SNP site S₁ is prepared. Then, the sample solution containing the DNA 1 is added with a typing primer 7a, a reverse primer 7b, a DNA polymerase 8, and four types of deoxyribonucleoside triphosphates (hereinafter, abbreviated to “dNTPs”, where N is adenine (A), cytosine (C), guanine (G), or thymine (T)). Here, the double-stranded DNA 1 is formed by single-stranded DNAs 3 and 4 which are complementary to each other, the typing primer 7a is complementary to a portion of the DNA 3, and the reverse primer 7b is complementary to a portion of the DNA 4. A base located at the SNP site S₁ of the single-stranded DNA 3 is thymine (T) (and a corresponding base in the complementary strand, i.e., the DNA 4, is adenine (A)). In the step shown in FIG. 2A, as in the step of FIG. 1A, a sample solution containing a target double-stranded DNA 2 having an SNP site S₂ is prepared. Then, the sample solution containing the DNA 2 is added with the typing primer 7a, the reverse primer 7b, the DNA polymerase 8, and four types of dNTPs. Here, the double-stranded DNA 2 is formed by single-stranded DNAs 5 and 6 which are complementary to each other, the typing primer 7a is complementary to a portion of the DNA 5, excluding the SNP site S₂, and the reverse primer 7b is complementary to a portion of the DNA 6, excluding the SNP site S₂. A base located at the SNP site S₂ of the single-stranded DNA 5 is cytosine (C) (and a corresponding base in the complementary strand, i.e., the DNA 6, is guanine (G)). Although a countless number of different SNP sites are present in the actual genomic DNA or the like, the description herein is given on the premise that a region of the DNA 1 to which the typing primer 7a binds and a region of the DNA 2 to which the typing primer 7b binds are the same as each other except that the SNP site S₁ of the DNA 1 and the SNP site S₂ of the DNA 2 differ from each other in their base sequences.

Next, in the step shown in FIG. 1B, the DNA 1 is subjected to thermal denaturation or the like so as to be split into the single-stranded DNAs 3 and 4. Similarly, in the step shown in FIG. 2B, the DNA 2 is subjected to thermal denaturation or the like so as to be split into the single-stranded DNAs 5 and 6.

Then, in the step shown in FIG. 1C, temperature adjustment is carried out in such a manner as to cause the typing primer 7a and the reverse primer 7b to be in hybridization with the single-stranded DNA 4 and the single-stranded DNA 3, respectively. In this case, the typing primer 7a completely hybridizes to the single-stranded DNA 4 in a region from the SNP site S₁ (which is adenine (A) in this example) toward the 3′ terminal. Similarly, in the step shown in FIG. 2C, temperature adjustment is carried out in such a manner as to cause the typing primer 7a and the reverse primer 7b to be in hybridization with the single-stranded DNA 6 and the single-stranded DNA 5, respectively. In this case, the typing primer 7a, excluding the 3′ terminal base T, hybridizes to the single-stranded DNA 6 in a region which is adjacent to the SNP site S₂ (which is guanine (G) in this example) on the 3′ terminal side of the single-stranded DNA.

Next, in the step shown in FIG. 1D, temperature adjustment is carried out in such a manner as to induce progress of a primer extension reaction. Since the typing primer 7a is in complete hybridization with the single-stranded DNA 4, which has A at the SNP site S₁, the primer extension reaction progresses, so that the dNTPs are consumed by the DNA polymerase 8.

Similarly, in the step shown in FIG. 2D, temperature adjustment is carried out in such a manner as to induce progress of a primer extension reaction. However, the typing primer 7a is in hybridization with the single-stranded DNA 6, which has G at the SNP S₂, in such a state where only the 3′ terminal base T thereof is not in hybridization, and therefore the primer extension reaction is unlikely to progress normally.

FIG. 3 is a diagram showing results of an electrophoretic analysis performed on DNA fragments contained in reaction solutions respectively having gone through the steps shown in FIGS. 1A-1D and the steps shown in FIGS. 2A-2D. In the example described in conjunction with FIGS. 1A-1D, the primer extension reaction of the typing primer 7a progresses satisfactorily, and therefore as shown in lane 1 of FIG. 3, a band representing a target base sequence is detected at the location indicated by arrow A. On the other hand, in the example described in conjunction with FIGS. 2A-2D, the primer extension reaction of the typing primer 7a progresses poorly, and therefore as shown in lane 2 of FIG. 3, a band representing a target base sequence is hardly detected at the location indicated by arrow A. Based on this result, it is possible to type the base at the SNP site S₁.

As described above, the SNP typing technique using the typing primer does not require any complicated operation or any specialized device, and therefore is deemed as one of the most effective among currently known SNP typing techniques. In the SNP typing technique using the typing primer, the design of the typing primer is most important. The typing primer is required to be designed such that the difference in progress of the primer extension reaction clearly appears with satisfactory reproducibility in accordance with the type of a base at the SNP site. Accordingly, a typing primer, which clarifies the difference in progress of the primer extension reaction with satisfactory reproducibility, has been actively developed.

As in the case of the typing primer 7a shown in FIGS. 1A through 2D, the most typical typing primer is designed so as to completely hybridize to a base sequence of a target single-stranded nucleic acid, which is adjacent to the SNP site on the 3′ terminal side of the target single-stranded nucleic acid, and so as to make a difference in progress of the primer extension reaction in accordance with a relationship between the type of a base at the 3′ terminal of the typing primer and the type of a base at the SNP site of the target single-stranded nucleic acid.

However, the typing primer used in the method described in conjunction with FIGS. 1A through 2D is not sufficiently usable to provide accurate SNP typing. In the case as shown in FIG. 2C, although the base at the 3′ terminal of the typing primer is in such a state as to be unable to hybridize to the SNP site S₂, if conditions of reaction temperature and time are not strictly controlled, the primer extension reaction may progress with the same degree of efficiency as in the case where the typing primer is in complete hybridization. In such a case, accurate SNP typing cannot be provided.

Accordingly, a recently developed typing primer includes a sequence complementary to a region of a specific sequence of a target nucleic acid, such that the 3′ terminal of the typing primer corresponds to the SNP site of the target nucleic acid and a base second from the 3′ terminal of the typing primer is uncomplementary to the sequence of the target nucleic acid (note that in a strict sense, the typing primer described above is distinct from the typing primer described in paragraph [0007] but referred to here as the typing primer because they function in the same manner; see, for example, Japanese Patent Laid-Open Publication No. 2002-101899). In this typing primer, if a base at a site corresponding to the SNP site (hereinafter, referred to as an “SNP corresponding site”) is complementary to the base at the SNP site of the target nucleic acid, the 3′ terminal of the typing primer can hybridize to the target nucleic acid, so that the primer extension reaction progresses. On the other hand, if the base at the SNP corresponding site is not complementary to the base at the SNP site of the target nucleic acid, two bases counted from the 3′ terminal of the typing primer are uncomplementary to the target nucleic acid, and therefore it becomes more difficult for the primer extension reaction to progress than in the case shown in FIG. 2C. Accordingly, a clear distinction can be made between a case where the primer extension reaction progresses and a case where the primer extension reaction does not progress, and more accurate SNP typing can reportedly be provided.

As another example of the recently developed typing primer, there is an Allele Specific Primer (ASP) developed by Toyobo Co., Ltd. (see International Publication WO01/042498 pamphlet). The ASP is designed such that the 3′ terminal base thereof is complementary to a base which is adjacent to a target SNP site of the target nucleic acid on the 5′ terminal side thereof, a base second from the 3′ terminal of the ASP corresponds to the target SNP site, and the third base from the 3′ terminal of the ASP is uncomplementary to a corresponding base of the target nucleic acid. It is reported that by providing a primer extension reaction using the ASP together with an alpha-type DNA polymerase having a high proofreading activity, it is made possible to accurately determine the type of abase at the SNP site as compared to the method described in conjunction with FIGS. 1A through 2D.

Specifically, consider a case where the method described in conjunction with FIGS. 1A through 2D provides the primer extension reaction using the ASP in place of the typing primer 7a and the alpha-type DNA polymerase having a high proofreading activity as the DNA polymerase 8. In this case, as shown in FIG. 4A, when a base (T) second from the 3′ terminal of an ASP 100 is complementary to abase (A) at the SNP site S₁ of the single-stranded DNA 4, the primer extension reaction progresses satisfactorily. On the other hand, as shown in FIG. 4B, when the base (T) second from the 3′ terminal of the ASP 100 is uncomplementary to a base (G) at the SNP site S₂ of the single-stranded DNA 6, two bases respectively second and third from the 3′ terminal of the ASP 100 are uncomplementary to the target nucleic acid, and therefore the primer extension reaction substantially does not progress. As a result, the difference in progress of the primer extension reaction is reportedly widened as compared to the method as described in conjunction with FIGS. 1A through 2D.

### SUMMARY OF THE INVENTION

Therefore, an object of the present invention is to provide: a base type determination method capable of determining the type of a target base in a nucleic acid with more accuracy and more satisfactory reproducibility; and a primer and kit for use in such a method.

One aspect of the present invention is directed to a base type determination method for determining a base type of a monobasic substituted region of a target nucleic acid. The base type determination method includes the steps of: (a) preparing a solution containing a target double-stranded nucleic acid having the monobasic substituted region, a base type determination primer, a DNA polymerase, and dNTPs; (b) causing the base type determination primer to hybridize to the target double-stranded nucleic acid in the solution, and causing a primer extension reaction to start progressing from the base type determination primer; and (c) analyzing the degree of progress of the primer extension reaction to determine the base type of the substituted region, the base type determination primer consisting of a first single-stranded nucleic acid which is capable of, when hybridizing to the target double-stranded nucleic acid, hybridizing to one of two strands of the target double-stranded nucleic acid such that a 3′ terminal of the primer corresponds to the substituted region of said one strand of the target double-stranded nucleic acid, the first single-stranded nucleic acid consisting of: a substitution corresponding region which is located at the 3′ terminal and consists of one base complementary to any one of predictable types of bases in the substituted region of said one strand of the target double-stranded nucleic acid; an uncomplementary region which is located adjacent to the substitution corresponding region on the 5′ terminal side thereof and consists of two bases uncomplementary to said one strand of the target double-stranded nucleic acid; and a complementary region which is located adjacent to the uncomplementary region on the 5′ terminal side thereof and is complementary to said one strand of the target nucleic acid, the complementary region consisting of a sufficient number of bases to hybridize to said one strand of the target nucleic acid under such conditions that the primer extension reaction of the first single-stranded nucleic acid can occur at least when a base in the substituted region is complementary to a base in the substitution corresponding region. Note that the substitution corresponding region consists of, most preferably, a single base, the uncomplementary region consists of, most preferably, two bases, and the complementary region consists of, preferably, five or more bases.

In the base type determination method of the present invention, when the base type determination primer is applied to a target single-stranded nucleic acid including a substituted region in which a base of one type might be substituted by a base of another type, the complementary region of the primer hybridizes to the target single-stranded nucleic acid. However, the uncomplementary region of the primer cannot hybridize to the single-stranded nucleic acid. In the case where a base in the substitution corresponding region is complementary to a base in the substituted region of the single-stranded nucleic acid, the substitution corresponding region hybridizes to the substitution region of the target single-stranded nucleic acid. On the other hand, in the case where a base in the substitution corresponding region is uncomplementary to a base in the substituted region of the target single-stranded nucleic acid, the substitution corresponding region cannot hybridize to the substitution region of the target single-stranded nucleic acid.

In the case where, although the uncomplementary region of the base type determination primer is in a state apart from the target single-stranded nucleic acid, the substitution corresponding region of the base type determination primer is in hybridization with the target single-stranded nucleic acid, a DNA polymerase, which is an enzyme for extending a nucleic acid from the 3′ terminal thereof, is able to normally work on the 3′ terminal of the base type determination primer.

On the other hand, in the case where neither the uncomplementary region nor the substitution corresponding region of the base type determination primer is able to hybridize to the target single-stranded nucleic acid, so that the uncomplementary region and the substitution corresponding region are in a state apart from the target single-stranded nucleic acid, the DNA polymerase is not able to normally work on the 3′ terminal of the base type determination primer.

Accordingly, when a base in the substitution corresponding region is complementary to a base in the substituted region of the target single-stranded nucleic acid and therefore hybridizes to the substituted region of the target single-stranded nucleic acid, the primer extension reaction occurs satisfactorily. However, when a base in the substitution corresponding region is uncomplementary to a base in the substituted region of the target single-stranded nucleic acid and therefore cannot hybridize to the substituted region of the target single-stranded nucleic acid, the primer extension reaction does not occur satisfactorily.

Therefore, there arises a clear difference in progress of the primer extension reaction between the case where the substituted region of the target single-stranded nucleic acid is complementary to the substitution corresponding region of the base type determination primer and the case where the substituted region of the target single-stranded nucleic acid is uncomplementary to the substitution corresponding region of the base type determination primer.

Accordingly, by analyzing the difference in progress of the primer extension reaction between the two cases, it is made possible to determine the type of a base in the substituted region of the target single-stranded nucleic acid. The present invention is also applicable to a case where there are two or more substituted bases in the substituted region of the target nucleic acid.

Preferably, the DNA polymerase has substantially no 3′→5′ exonuclease activity. The use of a DNA polymerase having substantially no 3′→5′ exonuclease activity eliminates a possibility such that the primer extension reaction might progress after nucleotides in both the substitution corresponding region and the uncomplementary region are cut, even if the substitution corresponding region of the base type determination primer has a base uncomplementary to a base in a substituted region of the target single-stranded nucleic acid and therefore cannot hybridize to the substituted region of the target single-stranded nucleic acid. Accordingly, it is possible to eliminate a possibility such that the type of a base in the substituted region of the target nucleic acid might not be accurately determined.

It is preferred that the base type determination primer is made of DNA. This is because the DNA is extremely chemically stable, and can be readily handled and obtained.

In a preferable embodiment of the present invention, in the step (a), the solution may further contain a reverse primer consisting of a single-stranded nucleic acid capable of hybridizing to the other strand of the target double-stranded nucleic acid, and in the step (b), the primer extension reaction may be caused to progress using a base sequence amplifying method selected from the group consisting of a PCR, an SDA, an RCR, an LAMP, and a TMA. Moreover, in the step (c), the base type of the substituted region may be determined based on a difference in progress of the primer extension reaction.

Preferably, in the step (c), the degree of progress of the primer extension reaction may be analyzed by using a method selected from the group consisting of electrophoresis, mass analysis, and liquid chromatography to measure the amount of amplification of a base sequence amplified by the base sequence amplifying method.

Preferably still, in the step (c), the degree of progress of the primer extension reaction may be analyzed by measuring the amount of pyrophosphoric acid generated by the primer extension reaction.

Preferably still, in the step (c), the amount of amplification of a base sequence amplified by the base sequence amplifying method may be measured by measuring the amount of pyrophosphoric acid generated by the primer extension reaction.

Preferably still, in the step (c), the amount of amplification of a base sequence amplified by the base sequence amplifying method may be quantitatively analyzed to determine the base type of the substituted region. Such a quantitative analysis is suitable for determining whether a base in the substituted region is the same (homo) or different (hetero) between a paternally-derived gene and a maternally-derived gene.

In another preferable embodiment, measurement of the amount of pyrophosphoric acid includes the steps of: converting the pyrophosphoric acid into an inorganic phosphoric acid within a sample containing at least a portion of the solution resulted from the step (b); providing the sample to a measurement system including glyceraldehyde 3-phosphate, oxidized nicotinamide adenine dinucleotide, glyceraldehyde 3-phosphatedehyrogenase, and at least one electron-transfer mediator; and measuring a value of current generated in the measurement system, and the value of current indicates a concentration of the pyrophosphoric acid in the sample.

Preferably, said at least one electron-transfer mediator is selected from the group consisting of ferricyanide, 1,2-naphthoquinone-4-sulfonic acid, 2,6-dichlorophenol-indophenol, dimethylbenzoquinone, 1-methoxy-5-methylphenazinium sulfate, methylene blue, gallocyanine, thionine, phenazine methosulfate, and meldora blue. More preferably, the measurement system further includes diaphorase.

Preferably still, the pyrophosphoric acid is converted into the inorganic phosphoric acid by causing the pyrophosphoric acid to react with pyrophosphatase in the sample.

In still another preferable embodiment, measurement of the amount of the pyrophosphoric acid includes the steps of: placing a sample including at least a portion of a solution resulted from the step (b) in one region of a measurement system having at least two regions divided by a membrane which holds H⁺-pyrophosphatase and has a limited permeability to H⁺; and measuring a change in concentration of H⁺ in either one of said at least two regions of the measurement system, and the degree of the change in concentration of H⁺ indicates the concentration of the pyrophosphoric acid in the sample.

In still another preferable embodiment, the measurement of the pyrophosphoric acid includes the steps of: providing the sample including at least a portion of a solution resulted from the step (b) to a measurement system including an artificial or natural membrane vesicle containing H⁺-pyrophosphatase therein; and measuring the change in concentration of H⁺ in the inside or outside of the membrane vesicle, and the degree of the change in concentration of H⁺ indicates the concentration of the pyrophosphoric acid in the sample. H⁺-pyrophosphatase provided in the measurement system is not limited to the form being enclosed in the above-mentioned spherical membrane such as a membrane vesicle, and a planar membrane, e.g., a plane membrane formed on an electrode, can also be used.

Preferably, the change in concentration of H⁺ is measured by either a method which measures an optical change converted from the change in concentration of H⁺ or a method which measures an electrical change converted from the change in concentration of H⁺.

Preferably still, the method which measures an optical change uses a pH test paper, a pH-sensitive dye, or a membrane potential-sensitive dye.

Preferably still, the method which measures an electrical change is selected from the group consisting of a metal electrode method, a glass electrode method, an ISFET electrode method, a patch-clamp method, and an LAPS method.

Preferably still, the method which measures an optical change uses the pH-sensitive dye to measure the change in concentration of H⁺ in the inside of the membrane vesicle.

In the base type determination primer, it is preferred that the total of the number of bases included in the substitution corresponding region and the number of bases included in the uncomplementary region is three or more.

In a preferable embodiment, the substitution corresponding region of the base type determination primer consists only of the 3′ terminal base of the single-stranded nucleic acid.

In still another preferable embodiment of the base type determination method of the present invention, in the step (a), the solution further contains a second base type determination primer; the second base type determination primer consists of a second single-stranded nucleic acid capable of, when hybridizing to the target double-stranded nucleic acid, hybridizing to one of two strand of the target double-stranded nucleic acid which is the same strand as that to which the first base type determination primer is supposed to hybridize, such that a 3′ terminal of the second base type determination primer corresponds to the substituted region of said one strand; and the second single-stranded nucleic acid includes: a second substitution corresponding region located at the 3′ terminal and consisting of one base which is complementary to any one of predictable types of bases in the substituted region of the target double-stranded nucleic acid and is different in type from said one base of the substitution corresponding region of the first single-stranded nucleic acid; a second uncomplementary region which is adjacent to the second substitution corresponding region on the 5′ terminal side and consists of two bases uncomplementary to said one strand of the target double-stranded nucleic acid; and a second complementary region which is adjacent to the second uncomplementary region on the 5′ terminal side and is complementary to said one strand of the target double-stranded nucleic acid, the second complementary region consisting of a sufficient number of bases to hybridize to said one strand of the target nucleic acid under such conditions that the primer extension reaction of the second single-stranded nucleic acid can occur at least when a base in the substituted region of the target nucleic acid is complementary to a base in the second substitution corresponding region. The second substitution corresponding region consists of, most preferably, one base, the second uncomplementary region consists of, most preferably, two bases, and the second complementary region consists of, preferably, five or more bases.

In the above embodiment, it is preferred that first single-stranded nucleic acid and the second single-stranded nucleic acid are different in length from each other.

In the above embodiment, it is further preferred that the first single-stranded nucleic acid and the second single-stranded nucleic acid are labeled by their respective fluorescences which are different in wavelength.

Another aspect of the present invention is directed to a base type determination primer for determining a base type of a monobasic substituted region of a target nucleic acid. The base type determination primer of the present invention consists of a single-stranded nucleic acid which is capable of hybridizing to the target nucleic acid such that a 3′ terminal of the primer corresponds to the substituted region of the target nucleic acid, and the single-stranded nucleic acid includes: a substitution corresponding region which is located at the 3′ terminal and consists of one base complementary to any one of predictable types of bases in the substituted region of the target nucleic acid; an uncomplementary region which is located adjacent to the substitution corresponding region on the 5′ terminal side thereof and consists of two bases uncomplementary to the target nucleic acid; and a complementary region which is located adjacent to the uncomplementary region on the 5′ terminal side thereof and is complementary to the target nucleic acid, the complementary region consisting of a sufficient number of bases such that the primer is able to hybridize to the target nucleic acid under such conditions that the primer extension reaction can progress. The substitution corresponding region consists of, most preferably, one base, the uncomplementary region consists of, most preferably, two bases, and the complementary region consists of, preferably, five or more bases.

In the base type determination primer of the present invention, it is preferred that the total of the number of bases included in the substitution corresponding region and the number of bases included in the uncomplementary region is three or more.

In a preferable embodiment of the base type determination primer of the present invention, the substitution corresponding region of the base type determination primer consists only of the 3′ terminal base of the single-stranded nucleic acid.

Still another aspect of the present invention is directed to a base type determination reagent kit for determining a base type of a monobasic substituted region of a target nucleic acid. The base type determination reagent kit includes a base type determination primer, a DNA polymerase, and dNTPs. In the base type determination reagent kit of the present invention, the primer consists of a first single-stranded nucleic acid which is capable of hybridizing to the target nucleic acid such that a 3′ terminal of the primer corresponds to the substituted region of the target nucleic acid, and the first single-stranded nucleic acid includes: a substitution corresponding region which is located at the 3′ terminal and consists of one base complementary to any one of predictable types of bases in the substituted region of the target nucleic acid; an uncomplementary region which is located adjacent to the substitution corresponding region on the 5′ terminal side thereof and consists of two bases uncomplementary to the target nucleic acid; and a complementary region which is located adjacent to the uncomplementary region on the 5′ terminal side thereof and is complementary to the target nucleic acid, the complementary region consisting of a sufficient number of bases such that said single-stranded nucleic acid is able to hybridize to the target nucleic acid under such conditions that the primer extension reaction can progress. The substitution corresponding region consists of, most preferably, one base, the uncomplementary region consists of, most preferably, two bases, and the complementary region consists of, preferably, five or more bases.

In the above kit, it is preferred that the DNA polymerase has substantially no 3′→5′ exonuclease activity.

In the above kit, it is preferred that the first single-stranded nucleic acid is a DNA.

Preferably, the base type determination reagent kit further includes a reverse primer.

Preferably still, the base type determination reagent kit further includes pyrophosphatase.

Preferably still, the base type determination reagent kit further includes glyceraldehyde 3-phosphate, oxidized nicotinamide adenine dinucleotide, glyceraldehyde 3-phosphatedehyrogenase, and at least one electron-transfer mediator. More preferably, the base type determination reagent kit further includes diaphorase.

Preferably still, said at least one electron-transfer mediator is selected from the group consisting of ferricyanide, 1,2-naphthoquinone-4-sulfonic acid, 2,6-dichlorophenol-indophenol, dimethylbenzoquinone, 1-methoxy-5-methylphenazinium sulfate, methylene blue, gallocyanine, thionine, phenazine methosulfate, and meldora blue.

Preferably still, the base type determination reagent kit further includes H⁺-pyrophosphatase. More preferably, the base type determination reagent kit further includes a pH test paper, a pH-sensitive dye, or a membrane potential-sensitive dye.

In still another preferable embodiment, the base type determination reagent kit of the present invention further includes a second base type determination primer, the second base type determination primer consisting of a second single-stranded nucleic acid capable of hybridizing to the target nucleic acid such that the 3′ terminal corresponds to the substituted region of the same strand as that to which the first base type determination primer is supposed to hybridize, and the second single-stranded nucleic acid including: a second substitution corresponding region located at the 3′ terminal and consisting of one base which is complementary to any one of predictable types of bases in the substituted region of the target nucleic acid and is different in type from said one base of the substitution corresponding region of the first single-stranded nucleic acid; a second uncomplementary region which is located adjacent to the second substitution corresponding region on the 5′ terminal side thereof and consists of two bases uncomplementary to the target nucleic acid; and a second complementary region which is adjacent to the second uncomplementary region on the 5′ terminal side and is complementary to the target nucleic acid, the second complementary region consisting of a sufficient number of bases to hybridize to the target nucleic acid under such conditions that the primer extension reaction of the second single-stranded nucleic acid can occur at least when a base in the substituted region of the target nucleic acid is complementary to a base in the second substitution corresponding region. The second substitution corresponding region consists of, most preferably, one base, the second uncomplementary region consists of, most preferably, two bases, and the second complementary region consists of, preferably, five or more bases.

Preferably, the first single-stranded nucleic acid and the second single-stranded nucleic acid are different in length from each other.

Preferably still, the first single-stranded nucleic acid and the second single-stranded nucleic acid are labeled by their respective fluorescences which are different in wavelength.

Preferably still, the base type determination reagent kit of the present invention may further include instructions specifying procedures for using the reagent to determine a base type and other information about precautions. More preferably, the base type determination reagent kit of the present invention may further include other reagents required for use in amplification reaction of a target base sequence or a quantitative analysis of an amplified base sequence.

(Definition of terms)

In the present specification, the term “target nucleic acid” refers to a single- or double-stranded nucleic acid having abase or bases targeted for analysis which is/are typically genomic DNA(s) or any fragment(s) thereof or may be a ribonucleic acid(s) (RNA(s)). For example, the target nucleic acid is an Alu sequence, or an exon, intron or promoter of a gene encoding a protein. Further, examples of the target nucleic acid include genes related to a variety types of diseases (including a genetic disease), drug metabolism, and a life-style related disease (e.g., hypertension, diabetes, etc.), and fragments of such genes. Furthermore, examples of the target nuclear acid may include portions in a genomic DNA other than gene portions or gene-related regions as described above. The target nucleic acid may be extracted from biological liquid (e.g., blood, serum, plasma, saliva, lymph, semen, vaginal mucosa, feces, urine, or spinal liquid) or biological tissue (e.g., hair or skin). Alternatively, the target nucleic acid can be extracted from a cell culture, a plant, food, a forensic sample (e.g., paper, fiber, scrap, water, sewage, and drug), etc. A method for use in extracting such a target nucleic acid is well-known to those skilled in the art.

The term “target single-stranded nucleic acid”, when used for describing the present invention, may mean either one of two strands of a target double-stranded nucleic acid.

In the present specification, the term “substituted region”, when used for describing the target nucleic acid, mainly means, but not limited to, a single nucleotide polymorphism site in the target nucleic acid, and examples of the substituted region may include a monobasic region or a region of two or more bases in the target nucleic acid where one or more bases are substituted or suspected of being substituted due to mutation.

In the present specification, the term “single nucleotide polymorphism”, or “SNP”, is used to mean what is normally meant in the field.

In the present specification, the term “allele”, or “allelomorph”, is used to mean what is normally meant in the field.

In the present specification, the term “substitution corresponding region” refers to a region of a base type determination primer of the present invention which includes a base(s) opposed to a base(s) in a substituted region of the target nucleic acid when the primer is in hybridization with the target nucleic acid. In the present invention, the substitution corresponding region of the base type determination primer typically consists of a single base at the 3′ terminal thereof which is complementary to any one of predictable types of bases in the substituted region of the target nucleic acid.

In the present specification, the term “uncomplementary region” refers to a region of the base type determination primer of the present invention which is located adjacent to the substitution corresponding region on the 5′ terminal side thereof, and the uncomplementary region includes bases opposed to bases in a region of the target nucleic acid which is located adjacent to the substituted region on the 3′ terminal side thereof when the primer is in hybridization with the target nucleic acid. In the present invention, the uncomplementary region of the base type determination primer consists of at least two bases which are uncomplementary to corresponding bases of the target nucleic acid, and most preferably the uncomplementary region consists of only two such bases.

In the present specification, the term “complementary region” refers to a region of the base type determination primer of the present invention which is located adjacent to the uncomplementary region on the 5′ terminal side thereof, and the region is complementary to and in hybridization with corresponding bases of the target nucleic acid when the primer is in hybridization with the target nucleic acid. In the present invention, the complementary region of the base type determination primer has a sufficient length such that the primer is able to hybridize to the target nucleic acid under the conditions that the primer extension reaction progresses, if at least abase in the substituted region of the target nucleic acid and a base in the substitution corresponding region of the base type determination primer is complementary to each other. Typically, the length of the complementary region corresponds to five or more bases.

(Effects of the Invention)

The present invention having the above-described features provides accurate and reproducible determination of a base type of a site of a target nucleic acid which is previously known as having a substituted base thereat. The present invention is advantageous, particularly for determining a base type of a single nucleotide polymorphism site of the target nucleic acid and for determining a base type of a site in which a base is substituted due to mutation or the like.

The base type determination primer of the present invention has at the 3′ terminal side thereof the uncomplementary region consisting of at least two bases, and therefore in the case where the substitution corresponding region of the primer consists of abase(s) uncomplementary to abase(s) in the substituted region of the target nucleic acid, a total of at least three bases at the 3′ terminal of the primer cannot hybridize to and therefore can be in a state apart from the target nucleic acid. In this case, as described later, substantially no primer extension reaction occurs, and therefore there arise a further clearer difference in progress of the primer extension reaction with respect to a case where the substitution corresponding region of the primer is complementary to a base in the substituted region of the target nucleic acid (in this case, the primer extension reaction progresses). In this manner, by using the primer of the present invention, the primer extension reaction can be curbed to such an extent as to allow substantially no reaction in the case where the primer extension reaction is not supposed to progress, whereby it is possible to provide an extremely clear distinction between a case where the nucleic acid is amplified and a case where the nucleic acid is not amplified. Accordingly, the present invention has an outstanding effect of providing a more accurate and reproducible determination of the type of a base in a region of the target nucleic acid which is substituted or suspected of being substituted.

Prior to the present invention, as far as the present inventors know, there are no instances of a base type determination which uses a primer having an uncomplementary region consisting of two or more bases (which exclude a case where the substitution corresponding region is uncomplementary to the substituted region of the target nucleic acid; this also applies to the following description). This is because it is a consensus view in the art that the primer extension reaction is unlikely to progress in the case where the primer has an uncomplementary region consisting of two or more bases uncomplementary to the target nucleic acid. In this context, the present invention is novel and may achieve unexpectedly considerable effects.

Further, as described in detail below, by using only one base type determination primer of the present invention to conduct a quantitative analysis of a base sequence amplified by a base sequence amplification method, it is possible to determine whether abase at an SNP site is the same (homo) or different (hetero) between a paternally-derived gene and a maternally-derived gene. As far as the present inventors know, there are no disclosures of any base type determination which uses only one base type determination primer. An effect of such a quantitative analysis of the amount of amplified base sequence can be further increased by using the primer of the present invention which provides a more accurate base type determination.

As describe above, the present invention provides an accurate and reproducible determination of the type of a target base in a nucleic acid. Further, the present invention can substantially unnecessitate, for example, an operation of strictly controlling reaction conditions, such as temperature and time conditions, for the primer extension reaction or an operation of changing the type of DNA polymerase to be used.

These and other objects, features, aspects and advantages of the present invention will become more apparent from the following detailed description of the present invention when taken in conjunction with the accompanying drawings.

### BRIEF DESCRIPTION OF THE DRAWINGS

FIGS. 1A-1D are stepwise views for explaining a conventional SNP typing technique;

FIGS. 2A-2D are stepwise views for explaining another conventional SNP typing technique;

FIG. 3 is a diagram showing results of an electrophoretic analysis performed on DNA fragments obtained by a conventional SNP typing technique;

FIGS. 4A and 4B are views each illustrating a structure of a conventional typing primer;

FIGS. 5A-5D are stepwise views for explaining a method for studying conditions under which a primer extension reaction is ensured not to progress;

FIG. 6 is a diagram showing results of an electrophoretic analysis performed on DNA fragments obtained in the study of conditions under which a primer extension reaction is ensured not to progress;

FIGS. 7A-7C are views each schematically illustrating a typing primer for use in a base type determination method according to a first embodiment;

FIGS. 8A-8D are views each illustrating a step of a method of the first embodiment which determines the type of a base in a base sequence of a substituted region included in a target single-stranded nucleic acid;

FIGS. 9A-9D are views each illustrating a step of a method of the first embodiment which determines the type of a base in a base sequence of a substituted region included in a target single-stranded nucleic acid;

FIGS. 10A-10C are schematic views each illustrating a structure of an SNP typing primer according to a second embodiment;

FIG. 11 is a diagram showing PCR reaction conditions;

FIG. 12 is a graph showing a relationship between the amount of pyrophosphoric acid contained in each PCR reaction liquid subjected to a PCR reaction and luminescence intensity;

FIG. 13 is a diagram showing PCR reaction conditions;

FIG. 14 is a graph showing a relationship between the amount of pyrophosphoric acid contained in each primer extension reaction liquid subjected to a primer extension reaction and luminescence intensity;

FIG. 15 is a diagram schematically illustrating H⁺-pyrophosphatase;

FIG. 16 is a diagram schematically illustrating a state of a solution containing H⁺-pyrophosphatase enclosed in a membrane vesicle;

FIG. 17 is a diagram illustrating a measurement device of pyrophosphoric acid using H⁺-pyrophosphatase enclosed in a plane membrane;

FIG. 18 is a graph showing a change of a fluorescence intensity of 540 nm for each PCR reaction liquid subjected to a PCR reaction;

FIG. 19 is a schematic view illustrating an exemplary measurement device system for measuring a current value; and

FIG. 20 is a graph showing a relationship between the amount of pyrophosphoric acid contained in each PCR reaction liquid subjected to a PCR reaction and a current value.

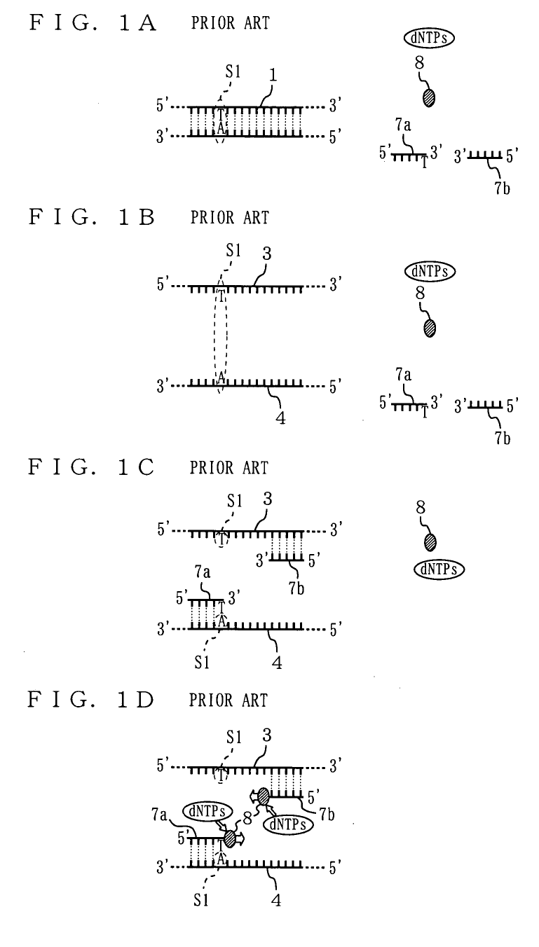

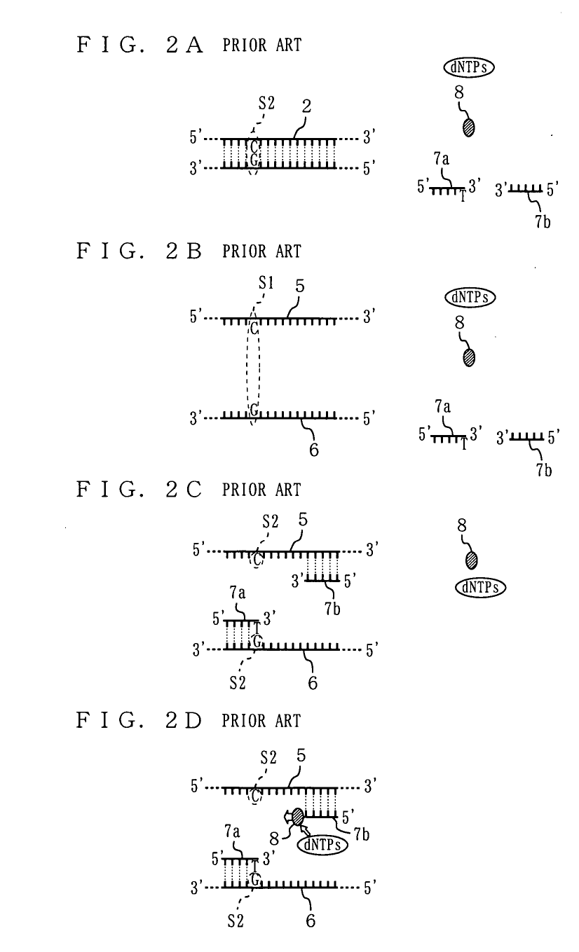

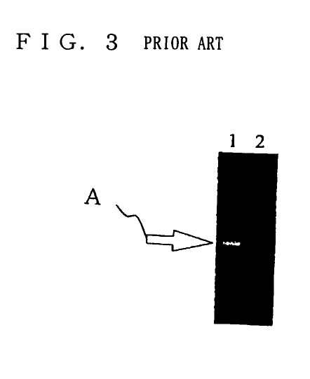

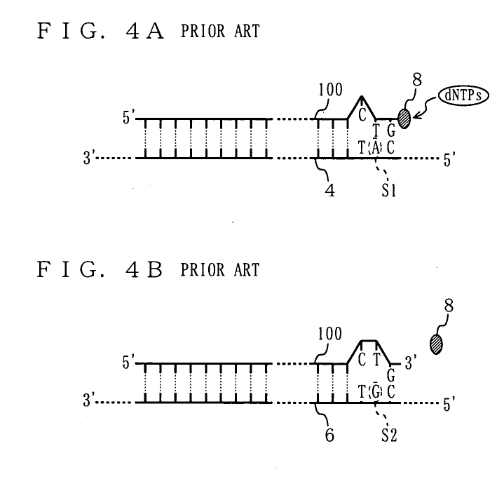

### DESCRIPTION OF THE PREFERRED EMBODIMENTS

(First Embodiment)

In some cases, the use of the ASP disclosed in International Publication WO01/042498 pamphlet does not result in sufficiently accurate SNP typing with satisfactory reproducibility. In the case where the primer extension reaction is not supposed to progress, even if the ASP is used, there remains a possibility that the primer extension reaction might occur. In order to eliminate such a possibility, some operations are required for more strictly controlling reaction conditions, such as temperature and time conditions, and/or for changing the type of a DNA polymerase to be used, for example. In some cases, however, even if such operations are repeatedly performed, SNP typing is still difficult to achieve.

Accordingly, the present inventors studied conditions under which the primer extension reaction is almost ensured not to progress. Specifically, a PCR method was used to study the correlation between the degree of the progress of the primer extension reaction and the number of bases at the 3′ terminal of a forward primer which are in such a state as to be unable to hybridize to a target single-stranded DNA.

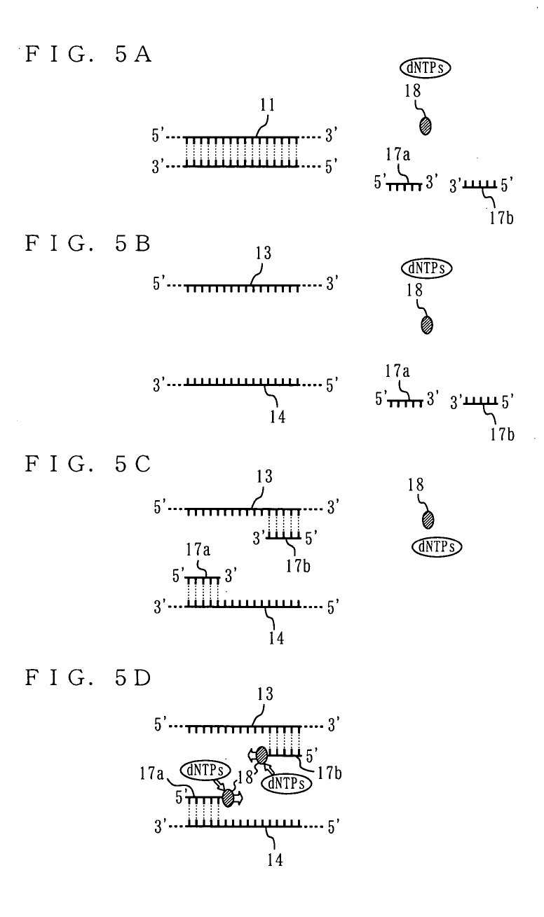

How the study was conducted is described with reference to FIGS. 5A-5D.

Firstly, in the step shown in FIG. 5A, a sample solution containing a target DNA 11 is prepared. Then, a forward primer 17a, a reverse primer 17b, a DNA polymerase 18, and four types of dNTPs are added to the sample solution containing the DNA 11.

Next, in the step shown in FIG. 5B, the DNA 11 is subjected to thermal denaturation or the like so as to be split into single-stranded DNAs 13 and 14.

Then, in the step shown in FIG. 5C, temperature adjustment is carried out in such a manner as to cause the forward primer 17a and the reverse primer 17b to be in hybridization with the single-stranded DNA 14 and the single-stranded DNA 13, respectively.

Next, in the step shown in FIG. 5D, temperature adjustment is carried out in such a manner as to cause a primer extension reaction to progress.

The above-mentioned steps shown in FIGS. 5B-5D are repeated to amplify DNA fragments initiated with the forward primer 17a and the reverse primer 17b.

In this study, λDNA is used as the DNA 11, and TaKaRa Taq (Takara Shuzo Co., Ltd.) having no 3′→5′ exonuclease activity is used as the DNA polymerase 18. For the details of the forward and reverse primers 17a and 17b used in the study, refer to Table 1 shown below and the attached sequence listing. As can be seen from Table 1, the reverse primer 17b (SEQ. ID No. 9) is consistently used with forward primers identified by SEQ. ID Nos. 1-8, and has a base sequence which can completely hybridize to the λDNA.

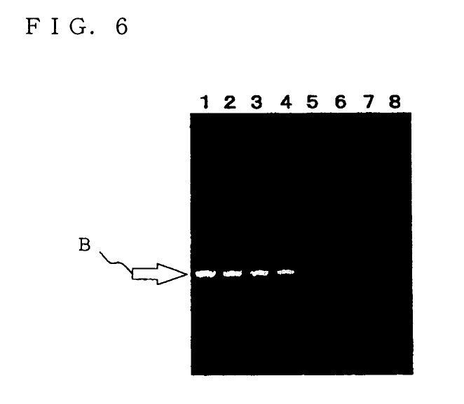

FIG. 6 shows results of electrophoresis which indicate the degrees of the progress of the primer extension reaction. Lane numbers 1-8 shown in FIG. 6 respectively correspond to SEQ. ID Nos. 1-8 shown in both Table 1 and the attached sequence listing.

TABLE 1

| Nos. | Forward Primer | Reverse Primer |
|---|---|---|
| 1 | λ300-1 (SEQ. ID No. 1) | λ300-2 (SEQ. ID No. 9) |
| 2 | λ300-1mA (SEQ. ID No. 2) | λ300-2 |
| 3 | λ300-1mT (SEQ. ID No. 3) | λ300-2 |
| 4 | λ300-1m2CT (SEQ. ID No. 4) | λ300-2 |
| 5 | λ300-1m2CA (SEQ. ID No. 5) | λ300-2 |
| 6 | λ300-1m3ACT (SEQ. ID No. 6) | λ300-2 |
| 7 | λ300-1m3ACA (SEQ. ID No. 7) | λ300-2 |
| 8 | λ300-1m3ACC (SEQ. ID No. 8) | λ300-2 |

When a forward primer specified by No. 1, which has a base sequence capable of completely hybridizing to the λDNA, was used, as shown in Lane 1 of FIG. 6, DNA fragments corresponding to the forward primer and the reverse primer were amplified in the location indicated by arrow B.

When a forward primer specified by No. 2 or 3, which has a base sequence including a base at the 3′ terminal incapable of hybridizing to the λDNA, was used, as shown in Lane 2 or 3 of FIG. 6, the same DNA fragment as that of Lane 1 was amplified.

Both forward primers specified by Nos. 4 and 5 have a base sequence including bases first and second from the 3′ terminal (i.e., two bases in total) incapable of hybridizing to the λDNA. When the forward primer specified by No. 5 was used, as shown in Lane 5 of FIG. 6, the same DNA fragment as that of Lane 1 was not amplified. However, when the forward primer specified by No. 4 was used, the same DNA fragment as that of Lane 1 was amplified. This indicates that the primer extension reaction may progress even if the two bases, i.e., the bases first and second from the 3′ terminal, are not in hybridization with a target sequence. This corresponds to a case where the 3′ terminal base of the typing primer disclosed in Japanese Laid-Open Patent publication No. 2002-101899 is uncomplementary to a base at the SNP site of the target nucleic acid, and the primer extension reaction is not supposed to progress. Accordingly, it is clear that even the use of the typing primer disclosed in Japanese Patent Laid-Open publication No. 2002-101899 is not sufficient for providing accurate SNP typing with satisfactory reproducibility.

Forward primers specified by Nos. 6-8 each have a base sequence including three bases at the 3′ terminal incapable of hybridizing to the λDNA. When the forward primers specified by Nos. 6, 7, and 8 were used, as shown in Lane 6, 7, and 8 of FIG. 6, the same DNA fragment as that of Lane 1 was not substantially amplified.

From the above results, it was confirmed that the primer extension reaction is almost ensured not to progress in a forward primer in which three bases at the 3′ terminal cannot hybridize to the target nucleic acid.

Accordingly, the present inventors concluded that in the case where SNP typing is carried out using the typing primer, the conditions under which the primer extension reaction is almost ensured not to progress is where three or more bases counted from the 3′ terminal of the typing primer do not hybridize to the target nucleic acid.

Hereinbelow, a first embodiment of the present invention will be described based on the above discussion and with reference to drawings.

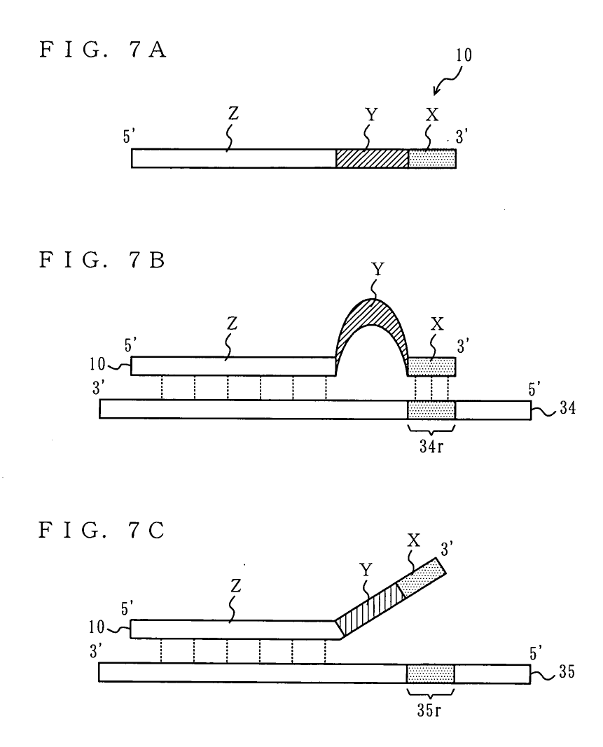

FIGS. 7A, 7B, and 7C are views each schematically illustrating a base type determination primer for use in a base type determination method according to the present embodiment (note that in a strict sense, the primer used here is distinct from the typing primer described in paragraph [0007] but referred to below as a typing primer because they function in the same manner).

A typing primer 10 of the present embodiment consists of a single-stranded nucleic acid capable of partially hybridizing to a target single-stranded nucleic acid including a substituted region which may include a substituted base. Here, it is premised that the location of the substituted region in the target nucleic acid is known. As shown in FIG. 7A, the single-stranded nucleic acid forming the typing primer 10 includes a substitution corresponding region X, an uncomplementary region Y, and a complementary region Z.

The substitution corresponding region X includes the 3′ terminal base of the single-stranded nucleic acid forming the typing primer 10 and also includes a base complementary to any one of predictable types of bases in the substituted region of the target single-stranded nucleic acid.

The uncomplementary region Y is located adjacent to the substitution corresponding region X on the 5′ terminal side thereof, and has a base sequence uncomplementary to the target single-stranded nucleic acid. The uncomplementary region Y consists preferably of at least two bases, most preferably of two bases.

The complementary region Z is located adjacent to the uncomplementary region Y on the 5′ terminal side thereof, and has a base sequence complementary to the target single-stranded nucleic acid. Preferably, the complementary region Z has a sufficient length to enable the typing primer 10 to hybridize to the single-stranded nucleic acid under such conditions that the primer extension reaction can occur.

The thus-configured typing primer of the present invention can be produced using an ordinary technique well-known to those skilled in the art. For example, the typing primer of the present invention can be produced using a commercially available DNA synthesizer, which can be readily obtained by those skilled in the art, in connection with a DNA synthetic method well-known in the art, such as a phosphoramidite method.

In the present specification, the term “primer” means a single-stranded nucleotide sequence including at least eight deoxyribonucleotides or ribonucleotides. Normally, the primer has a sufficient length and sufficient complementarity to the target nucleic acid such that the primer is able to hybridize to the target nucleic acid under such conditions that the primer extension reaction can progress. In the present invention, the length of the primer corresponds preferably to eight to one hundred bases, more preferably to ten to sixty bases, further more preferably to twelve to forty bases, still further more preferably to fourteen to thirty-five bases, still further more preferably to sixteen to thirty bases, most preferably to eighteen to twenty-five bases.

When the typing primer 10 according to the present embodiment is applied to each one of target single-stranded nucleic acids 34 and 35 each having a substituted region, which may include a substituted base, the target single-stranded nucleic acids 34 and 35 are brought into states as shown in FIGS. 7B and 7C, respectively. Note that the actual genomic DNA or the like includes a countless number of regions, such as SNP sites, which include a substituted base, but in the case as described here, it is premised that base sequences of the target single-stranded nucleic acids 34 and 35 are the same as each other in regions to which the typing primer 10 is bound, except that base sequences of substituted regions 34r and 35r are different from each other in base type.

As shown in FIGS. 7B and 7C, the complementary region Z hybridizes to the target single-stranded nucleic acids 34 and 35. On the other hand, as is apparent from FIGS. 7B and 7C, the uncomplementary region Y is not able to hybridize to the target single-stranded nucleic acids 34 and 35. In the case where the base sequence of the substitution corresponding region X is complementary to the base sequence of the substituted region 34r of the target single-stranded nucleic acid 34, as shown in FIG. 7B, the substitution corresponding region X hybridizes to the substituted region 34r of the target single-stranded nucleic acid 34. On the other hand, in the case where the base sequence of the substitution corresponding region X is not complementary to the base sequence of the substituted region 35r of the target single-stranded nucleic acid 35, as shown in FIG. 7C, the substitution corresponding region X is not able to hybridize to the substituted region 35r of the target single-stranded nucleic acid 35.

In the typing primer 10 shown in FIG. 7B, although the uncomplementary region Y is in a state apart from the target single-stranded nucleic acid 34, the substitution corresponding region X is in hybridization with the substituted region 34r of the target single-stranded nucleic acid 34. Therefore, a DNA polymerase, which is an enzyme for extending a nucleic acid from the 3′ terminal thereof, is able to normally work on the 3′ terminal of the typing primer 10.

On the other hand, in the typing primer 10 shown in FIG. 7C, neither the uncomplementary region Y nor the substitution corresponding region X of the typing primer 10 is able to hybridize to the target single-stranded nucleic acid 35 on which the typing primer 10 is applied, so that the uncomplementary region Y and the substitution corresponding region X are in a state apart from the target single-stranded nucleic acid 35. As a result, the DNA polymerase is not able to normally work on the 3′ terminal of the typing primer 10.

Accordingly, as shown in FIG. 7B, when the base sequence of the substitution corresponding region X is complementary to the base sequence of the substituted region 34r of the single-stranded nucleic acid 34 and therefore the substitution corresponding region X hybridizes to the substituted region 34r of the single-stranded nucleic acid 34, the primer extension reaction occurs satisfactorily. However, as shown in FIG. 7C, when the substituted region X has bases uncomplementary to the base sequence of the substituted region 35r of the single-stranded nucleic acid 35 and thus is unable to hybridize to the substituted region 35r of the single-stranded nucleic acid 35, the primer extension reaction does not occur satisfactorily.

Therefore, a difference in progress of the primer extension reaction is considerable between the case as shown in FIG. 7B, where the substituted region 34r of the single-stranded nucleic acid 34 is complementary to the substitution corresponding region X of the typing primer 10, and the case as shown in FIG. 7C, where the substituted region 35r of the single-stranded nucleic acid 35 is uncomplementary to the substitution corresponding region X of the typing primer 10.

Accordingly, by analyzing the difference in progress of the primer extension reaction between the two cases, it is made possible to determine the type of a base in each base sequence of the substituted region 34r of the single-stranded nucleic acid 34 and the substituted region 35r of the target single-stranded nucleic acid 35.

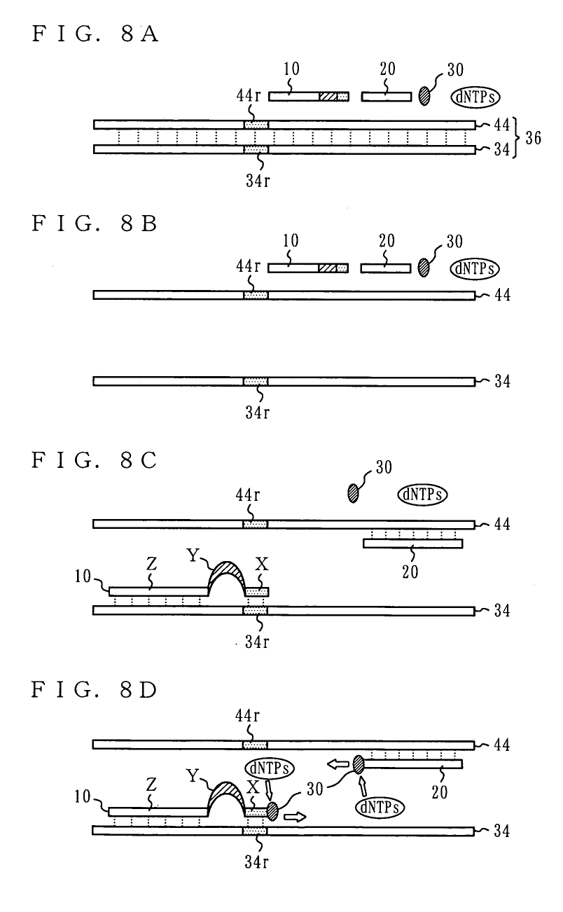

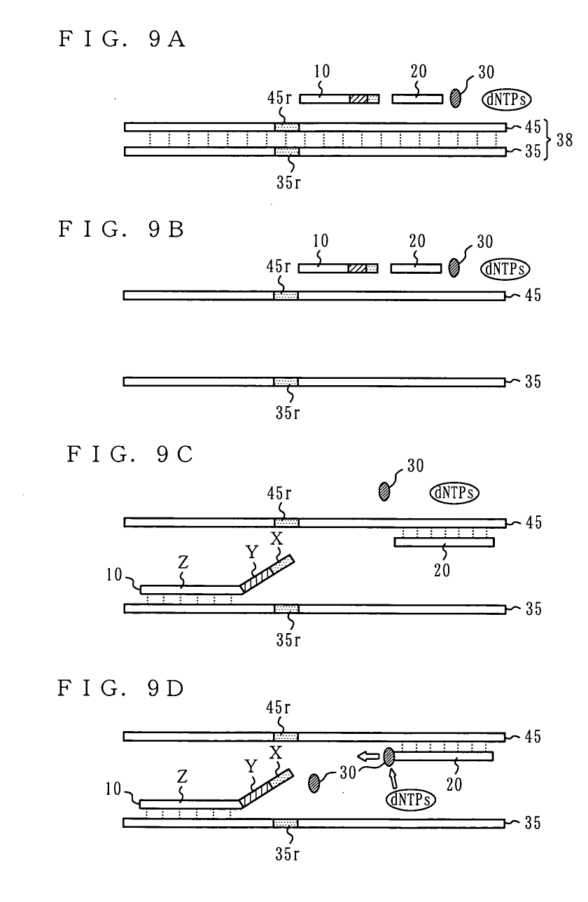

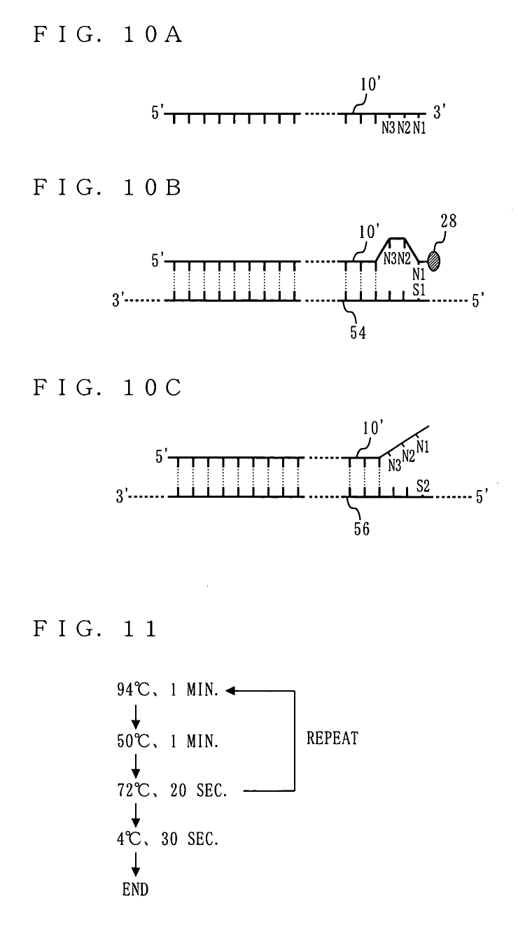

Referring to FIGS. 8A-8D and FIGS. 9A-9D, described next is a method which uses the typing primer 10 to determine the type of a base in a base sequence of a substituted region of a target double-stranded nucleic acid which may include a substituted base. Also, in this case, the location of the substituted region, which may include a substituted base, in the target nucleic acid is known. FIGS. 8A-8D and FIGS. 9A-9D are views each illustrating a step of a method of the present embodiment which uses a PCR to determine the type of a base in a base sequence of the substituted region included in a target double-stranded nucleic acid.

Firstly, the step shown in FIG. 8A prepares a sample solution containing a target nucleic acid 36 having substituted regions 34r and 44r. Then, the sample solution containing the nucleic acid 36 is added with the typing primer 10, a reverse primer 20, a DNA polymerase 30, and four types of dNTPs. Similarly, the step shown in FIG. 9A prepares a sample solution containing a target nucleic acid 38 having substituted regions 35r and 45r. Then, the sample solution containing the nucleic acid 38 is added with the typing primer 10, the reverse primer 20, the DNA polymerase 30, and four types of dNTPs. Note that an actual genomic DNA or the like includes a countless number of regions, such as SNP sites, which include a substituted base, but in the case as described here, it is premised that base sequences of the target single-stranded nucleic acids 36 and 38 are the same as each other in regions to which the typing primer 10 is bound except that substituted regions 34r and 44r are different from substituted regions 35r and 45r in base type.

Next, in the step shown in FIG. 8B, the nucleic acid 36 is subjected to thermal denaturation or the like so as to be split into target single-stranded nucleic acids 34 and 44. Similarly, in the step shown in FIG. 9B, the nucleic acid 38 is subjected to thermal denaturation or the like so as to be split into target single-stranded nucleic acids 35 and 45.

Next, in the step shown in FIG. 8C, temperature adjustment is carried out in such a manner as to cause the typing primer 10 and the reverse primer 20 to be in hybridization with the target single-stranded nucleic acids 34 and 44, respectively. In this case, the complementary region Z of the typing primer 10 hybridizes to the target single-stranded nucleic acid 34, and the substitution corresponding region X of the typing primer 10 hybridizes to the substituted region 34r of the target single-stranded nucleic acid 34. Similarly, in the step shown in FIG. 9C, temperature adjustment is carried out in such a manner as to cause the typing primer 10 and the reverse primer 20 to be in hybridization with the target single-stranded nucleic acids 35 and 45, respectively. In this case, only the complementary region Z of the typing primer 10 hybridizes to the target single-stranded nucleic acid 35.

Then, in the step shown in FIG. 8D, temperature adjustment is carried out again in such a manner as to induce a progress of a primer extension reaction. In this case, the substitution corresponding region X of the typing primer 10 is in hybridization with the substituted region 34r of the target single-stranded nucleic acid 34, and therefore the primer extension reaction progresses, so that the dNTPs are consumed by the DNA polymerase 30.

Similarly, in the step shown in FIG. 9D, temperature adjustment is carried out in such a manner as to induce a progress of a primer extension reaction. However, the substitution corresponding region X is not able to hybridize to the substituted region 35r of the target single-stranded nucleic acid 35, and therefore the primer extension reaction is unlikely to normally progress.

In the present embodiment, the steps shown in FIGS. 8B-8D and the steps shown in FIGS. 9B-9D are repeatedly performed based on a procedure of the PCR method.

After the above-described steps are performed, by analyzing a difference in progress of the primer reaction (in the present embodiment, especially, a difference in amount of amplified nucleic acids), it is made possible to determine the type of a base in the base sequence of the substituted region of the target single-stranded nucleic acid.

In view of the condition under which the primer extension reaction is almost ensured not to progress, it is preferred that in the step shown in FIG. 9C, the number of bases of the typing primer 10 which are not in hybridization (i.e., the total number of bases included in both the substitution corresponding region X and the uncomplementary region Y) is three or more. Accordingly, when the substitution corresponding region X is not able to hybridize to the substituted region 35r of the target single-stranded nucleic acid 35, the primer extension reaction is almost ensured not to occur. Therefore, a difference in progress of the primer extension reaction is distinctively clarified.

When the difference in progress of the primer extension reaction is considerable, an extremely clear difference can be seen in a result obtained by analyzing the difference in progress of the primer extension reaction. Accordingly, it is possible to more accurately determine whether the target nucleic acid has the same base sequence as the base sequence of the substituted region 34r or the base sequence of the substituted region 35r.

In the present embodiment, the PCR method is used to provide the primer extension reaction of the typing primer 10. However, the present invention is not limited to this, and any method for amplifying the nucleic acid having a specific base sequence, such as an SDA, an RCR, a LAMP, a TMA, or the like, can be used. Examples of the method for analyzing the difference in progress of the primer extension reaction include a method which uses electrophoresis or the like to analyze the nucleic acid amplified as described above, and a method which analyzes the amount of pyrophosphoric acid generated during the amplification of the nucleic acid as described above.

Although the present embodiment has been described with respect to the case where the reverse primer 20 is added, the present invention is not limited to this. For example, the primer extension reaction of the typing primer 10 may be provided without adding the reverse primer 20, and then the amount of pyrophosphoric acid generated during the primer extension reaction may be analyzed.

Any method can be used so long as the method is able to accurately analyze the difference in progress of the primer extension reaction.

For the nucleic acid forming the typing primer 10, DNA is preferred. This is because the DNA is extremely chemically stable, and can be readily handled and obtained. It goes without saying that the typing primer 10 can be produced using thiol DNA, RNA, or the like, as necessary.

In the case of using the dNTPs for the primer extension reaction, it is necessary to use a DNA polymerase as the nucleic acid polymerase. The DNA polymerase may or may not have a 3′→5′ exonuclease activity. In the present embodiment, it is preferred to use a DNA polymerase substantially having no 3′→5′ exonuclease activity (i.e., a DNA polymerase having no 3′→5′ exonuclease activity or a 3′→5′ exonuclease activity which is sufficiently weak and thus can be ignored from a measurement viewpoint; this is true of any DNA polymerase described below as substantially having no 3′→5′ exonuclease activity). The reason for this is that in the case of using the typing primer 10 of the present embodiment, as described above, the substitution corresponding region X is uncomplementary to the substituted region 35r of the target single-stranded nucleic acid 35, and therefore the substitution corresponding region X and the uncomplementary region Y of the typing primer 10 are not able to hybridize to the single-stranded nucleic acid and therefore is presumably left in a loosened state. In such a case, the DNA polymerase substantially having no 3′→5′ exonuclease activity normally cannot induce a normal primer extension reaction. However, when using a DNA polymerase having a high 3′→5′ exonuclease activity, the high activity may causes the primer extension reaction to progress after cutting nucleotides in the substitution corresponding region X and the uncomplementary region Y, making it impossible to accurately determine the type of a base in the substituted region of the target single-stranded nucleic acid.

Therefore, it is preferred to use the DNA polymerase substantially having no 3′→5′ exonuclease activity. Specific examples of such a DNA polymerase include a TaKaRa Taq (produced by Takara Shuzo CO., Ltd.), an rTaq DNA Polymerase (produced by Toyobo Co., Ltd.), a Taq DNA Polymerase (produced by Amersham Pharmacia Biotech), a Tfl DNA Polymerase (produced by Promega), a Hot Tub DNA Polymerase (produced by Amersham Pharmacia Biotech), a Tth DNA Polymerase (produced by Toyobo Co., Ltd.), an rTth DNA Polymerase (produced by Toyobo, PE Biosystems), and an Ampil Taq DNA Polymerase (e.g., a DNA Polymerase produced by Applied Biosystems). By using such a DNA polymerase, it is made possible to eliminate the above-described possibility of inaccurate determination, and therefore it is possible to more accurately determine the type of a base in the substituted region of the target single-stranded nucleic acid. Another advantage of using the DNA polymerase substantially having no 3′→5′ exonuclease activity is that such a DNA polymerase is available at a low cost. Further, as listed above, the DNA polymerase substantially having no 3′→5′ exonuclease activity is various in type and can be readily obtained.

The present invention can substantially unnecessitate, for example, an operation of strictly controlling reaction conditions, such as temperature and time conditions, for the primer extension reaction or an operation of changing the type of DNA polymerase to be used. For example, the present inventors have confirmed that the typing primer of the present invention extremely clearly presents a difference between a case where the primer extension reaction progresses and a case where the primer extension reaction does not progress even if an annealing temperature during a PCR cycle is set at any level within the range between about 50° C. and about 60° C.

(Second Embodiment)

In the present embodiment, among the typing primers 10 described in the first embodiment, a typing primer especially suited for SNP typing is described.

An SNP represents a difference (or a substitution) of a base pair observed at a specific site in a base sequence of a genomic DNA. Accurate SNP typing can be provided by using a typing primer 10 which includes only a 3′ terminal base as the substitution corresponding region X.

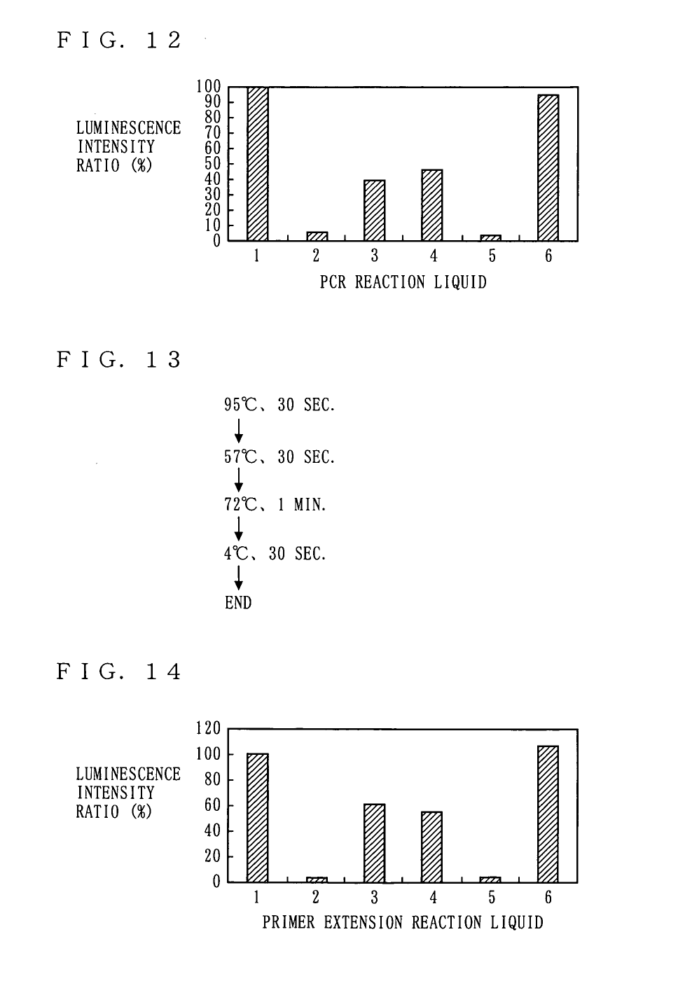

A typing primer used for SNP typing will now be described in detail with reference to FIGS. 10A-10C. FIGS. 10A-10C are views used for explaining the structure of the typing primer used for SNP typing.

A typing primer 10′ for use in SNP typing consists of a single-stranded nucleic acid. As the nucleic acid, DNA is preferable. This is because DNA is chemically stable and can be readily handled and obtained. It goes without saying that the typing primer 10′ can be produced using thiol DNA, RNA, or the like, as necessary. In the typing primer 10′, a base located at the 3′ terminal (shown as N₁ in the figures) corresponds to a base S₁ or S₂ at each SNP site of target single-stranded nucleic acids 54 and 56. It is premised that each SNP site of the target nucleic acids 54 and 56 is previously known. The typing primer 10′ is designed such that bases second and third from the 3′ terminal of the typing primer 10′ (respectively indicated by N₂ and N₃ in the figures) are always respectively uncomplementary to bases second and third from the SNP site toward the 3′ terminal direction of each of the target single-stranded nucleic acids 54 and 56, and a base sequence between a base fourth from the 3′ terminal of the typing primer 10′ and a 5′ terminal of the typing primer 10′ is complementary to a base sequence of fourth and following bases counted from the SNP site toward the 3′ terminal direction of each of the target single-stranded nucleic acid 54 and 56.

In this embodiment, it is assumed that the bases N₁ and S₁ are complementary to each other and the bases N₁ and S₂ are uncomplementary to each other. When dNTPs are used in the primer extension reaction, the DNA polymerase is used as a nucleic acid polymerase.

As shown in FIG. 10B, although the bases N₂ and N₃, respectively second and third from the 3′ terminal of the typing primer 10′, are not able to hybridize to the target single-stranded nucleic acid 54 having the base S₁ at the SNP site, the base N₁ at the 3′ terminal is able to hybridize to the base S₁ at the SNP site of the target single-stranded nucleic acid 54. Therefore, a DNA polymerase 28 is able to normally work on the 3′ terminal of the typing primer 10′, leading to a satisfactory progress of the primer extension reaction.

On the other hand, as shown in FIG. 10C, the base N₁ at the 3′ terminal of the typing primer 10′, and the bases N₂ and N₃, respectively second and third from the 3′ terminal of the typing primer 10′, are all not able to hybridize to the target single-stranded nucleic acid 56 having the base S₂ at the SNP site. Therefore, the DNA polymerase 28 is not able to normally work on the 3′ terminal of the typing primer 10′, so that the primer extension reaction is unlikely to normally progress (see Table 1 and FIG. 6).

Specifically, as shown in FIG. 10B, if the 3′ terminal base of the typing primer 10′ is in such a relationship with the base at the SNP site of the target single-stranded nucleic acid 54 as to be complementary to each other, the primer extension reaction occurs satisfactorily, while as shown in FIG. 10C, if the 3′ terminal base of the typing primer 10′ is uncomplementary to the base at the SNP site of the target single-stranded nucleic acid 56, the primer extension reaction substantially does not progress.

Accurate SNP typing can be provided by using the typing primer 10′ designed in a manner as described above, instead of using the typing primer 10, to provide the primer extension reaction under the same procedure as that described in relation to the method for determining the type of a base in a base sequence of the substituted region described in the first embodiment, and thereafter by analyzing a difference in progress of the primer extension reaction. Accordingly, by analyzing the difference in progress of the primer extension reaction, it is made possible to determine the type of the base in the base sequence of the SNP site of the target single-stranded nucleic acid.

Further, by using the typing primer according to the present embodiment, it is made possible to provide not only SNP typing but also analysis of a single-base variation due to a mutation. The present invention is not limited this, and it is also possible to determine the type of any single base desired to be typed.

(Third Embodiment)

A genome of a higher organism such as a human consists of both paternally-derived genes and maternally-derived genes. A pair of a paternally-derived gene and a maternally-derived gene is called an allele. In SNP typing, it is considerably important to determine whether the base at the SNP site of the paternally-derived gene and the base at the SNP site of the maternally-derived gene are the same (i.e. homo) or different (i.e. hetero). The typing primer according to the second embodiment is also applicable to such SNP typing.

Described next is an exemplary case where in a sample extracted from a human, a base at the SNP site of a target single-stranded nucleic acid is previously known to be thymine (T) or cytosine (C).

In this case, in a higher organism such as a human having a polyploid genome as described above, offspring's genes derived from the parents can be classified into three SNP patterns, i.e., T/T homo, C/C homo, and T/C hetero. In order to distinguish among these patterns, a PCR is carried out using a typing primer (hereinafter, referred to as a “typing primer A”) in which the primer extension reaction progresses only when the base at the SNP site is T, and a typing primer (hereinafter, referred to as a “typing primer B”) in which the primer extension reaction progresses only when the base at the SNP site is C. Note that each of the typing primers A and B is designed identical to the typing primer 10′ descried in the second embodiment, and a reverse primer identical to that described in the second embodiment is used.

Next, each DNA fragment is analyzed by electrophoresis or the like, whereby it is possible to type the SNP pattern.

Specifically, the SNP pattern can be determined as: T/T in the case where the DNA fragments are amplified only when the typing primer A is used; C/C in the case where the DNA fragments are amplified only when the typing primer B is used; and T/C in the case where the DNA fragments are amplified when either of the typing primers A and B is used.

Further, if the typing primers A and B are designed such that their respective complementary regions X are different in length from each other, a DNA fragment amplified by the typing primer A and the reverse primer has a length different from that of a DNA fragment amplified by the typing primer B and the reverse primer. Accordingly, even if electrophoresis is simultaneously performed on both of the DNA fragments, bands of the DNA fragments do not overlap with each other. Thus, the typing primers A and B are simultaneously mixed, a PCR is then carried out using the reverse primer, and thereafter both of the DNA fragments are simultaneously analyzed by electrophoresis or the like, thereby typing the SNP pattern rapidly.

Alternatively, if the typing primers A and B are labeled by their respective fluorescences which are different in wavelength, the DNA fragment amplified by the typing primer A and the reverse primer emits fluorescence with a wavelength different from that of fluorescence emitted by the DNA fragment amplified by the typing primer B and the reverse primer. Accordingly, it is possible to separately detect the individual DNA fragments. Thus, the typing primers A and B are simultaneously mixed, a PCR is then carried out using the reverse primer, and thereafter an amplified DNA fragment is isolated by electrophoresis or the like, and a fluorescent wavelength of the isolated DNA fragment is analyzed, thereby typing the SNP pattern rapidly. Examples of label reagents with different wavelengths include a combination of Cy3™ (Amersham Life Science Inc.) and Cy5™ (Amersham Life Science Inc.) and any combination of label reagents selected from the group consisting of JOE (having an emission peak at 550 nm), ROX (having an emission peak at 600 nm), FAM (having an emission peak at 520 nm) and TAMRA (having an emission peak at 580 nm). The above label reagents can be readily obtained as commercial products from suppliers, such as Invitrogen Corp. and Funakoshi Co. Ltd., which are well-known to those skilled in the art.

Further, it is also possible to carry out typing of the SNP patterns using only the typing primer A.

Specifically, a PCR is carried out using the typing primer A and the reverse primer, and thereafter analysis is carried out by electrophoresis or the like. The DNA fragment is amplified when the SNP pattern is either T/T or T/C, and is not substantially amplified when the SNP pattern is C/C. Moreover, in the case where the SNP pattern is T/T, the amount of the target single-stranded nucleic acid having T as a base at the SNP site used as a template in the PCR is twice as much as in the case where the SNP pattern is T/C, and therefore the degree of amplification of the DNA fragment is clearly increased. Accordingly, by analyzing the amount of amplification of the DNA fragment, it is made possible to achieve typing of the SNP pattern of the SNP site. Such discrimination between homo and hetero by quantitative analysis of the amount of amplified nucleic acid can be advantageously achieved by using the primer of the present application which clarifies a difference between a case where the DNA fragment is amplified and a case where the DNA is not amplified.

Even in the case where the number of base types which can be the type of a base at the SNP site is three or four, if three or four typing primers of the third embodiment are provided in accordance with the number of types of the bases, such that their respective bases N₁ at the 3′ terminal are different from each other (i.e., there are three or four different types of bases N₁), it is possible to achieve SNP typing.

(Fourth Embodiment)

As in the first through third embodiments, the genomic DNA and the typing primer are used for amplifying a nucleic acid by a base sequence amplification method such as a PCR. Thereafter, a resultant amplification reaction liquid is introduced into a measurement system including membrane vesicles each containing H⁺-pyrophosphatase therein, and a change in concentration of H⁺ caused in the inside or outside of a membrane vesicle is measured, thereby analyzing the amount of amplification of the nucleic acid, whereby it is made possible to determine the base type.

The H⁺-pyrophosphatase is a membrane protein normally present in a tonoplast of a plant, and has a property of transporting H⁺ from the outside of the tonoplast into the inside of the tonoplast, while providing a hydrolysis reaction which generates a bimolecular phosphoric acid from a monomolecular pyrophosphoric acid. By exploiting this property, natural or artificial membrane vesicles enclosing H⁺-pyrophosphatase are provided to a sample containing amplified nucleic acid, and a change in concentration of H⁺ in the inside or outside of a membrane is measured by, for example, an optical method which uses a pH test paper, a pH-sensitive dye (e.g., acridine orange), or a membrane potential-sensitive dye (e.g., DiBAC₄ (3) (Bis (1,3-dibutylbarbituric acid)trimethine oxonol), DiBAC₄ (5) (Bis (1,3-dibutylbarbituric acid)pentamethine oxonol), DiSBAC₂ (3) (Bis (1,3-diethylthiobarbituric acid)trimethine oxonol), di-4-ANNEPS, DiOC₆ (3) (dihexaoxacarbocyanine iodide), or oxonol V), or an electrochemical method such as a metal-electrode method (e.g., a hydrogen-electrode method, a quinhydrone-electrode method, or an antymony-electrode method), a glass-electrode method, an ion-selective field-effective transistor electrode (ISFET) method, a patch-clamp method, or a light-addressable potentiometric sensor (LAPS) method, thereby quantitatively measuring pyrophosphoric acid generated by amplification reaction of the nucleic acid. Based on the amount of the thus-measured pyrophosphoric acid, it is possible to carry out determination of the type of a base in the substituted region of the target nucleic acid as well as typing of the SNP site in the genomic DNA. Note that H⁺-pyrophosphatase may be enclosed not only in a spherical membrane such as a membrane vesicle but also in a plane membrane (formed on, for example, an electrode).

For example, in a pyrophosphoric acid measurement device 200 as shown in FIG. 17, which includes a container 204, an electrode 205, and an inner vessel 206 provided in the container 204, a planar membrane 207 containing H⁺-pyrophosphatase may be used. At the bottom of the inner vessel 206 of the pyrophosphoric acid measurement device 200, an H⁺-sensitive electrode 208 is provided, and active sites of H⁺-pyrophosphatase which hydrolyze pyrophosphoric acid are exposed to the outside of the inner vessel 206. When a sample solution 202 is injected into the container 204, if pyrophosphoric acid is present in the sample solution 202, an enzyme reaction of H⁺-pyrophosphatase occurs, so that the concentration of H⁺ increases in an inner region 209 of the inner vessel 206 which is separated by the membrane 207 from the outside of the inner vessel 206, while the concentration of H⁺ decreases in the outside of the inner vessel 206. Accordingly, it is possible to measure the amount of pyrophosphoric acid by electrically measuring a change in concentration of H⁺ using the electrode 205 and the H⁺-sensitive electrode 208. In the present embodiment, the sample solution 202 is previously injected into the container 204 and the inner region 209. However, the present invention is not limited to this, and the sample solution 202 may be added into the container 204 after providing the membrane 207 on the H⁺-sensitive electrode 208 in the inner vessel 206. In this case, if the sample solution 202 is injected into the container 204, some of components of the sample solution 202 which are transmitted through the membrane 207 (i.e., portions of the sample solution which do not include pyrophosphoric acid) fill the inner region 209, whereby it is made possible to electrically measure the change in concentration of H⁺ using the electrode 205 and the H⁺-sensitive electrode 208.

Note that the membrane 207 may include H⁺-pyrophosphatase having active sites which hydrolyze pyrophosphoric acid and is exposed to the inner region 209. However, in the case of using the membrane 207 including such H⁺-pyrophosphatase having active sites which hydrolyze pyrophosphoric acid and is exposed to the inner region 209, it is preferred to control the concentration of pyrophosphoric acid within the inner region 209 so as to be kept lower than that of pyrophosphoric acid in the outside of the inner vessel 206. It is most preferred that the inner region 209 does not include pyrophosphoric acid. In this case, transportation of H⁺ from the inner region 209 to the outside of the inner vessel 206 is reduced or stopped, so that H⁺ is predominantly transported from the outside of the inner vessel 206 into the inner region 209. As a result, the concentration of H⁺ in the outside of the inner vessel 206 and the concentration of H⁺ within the inner region 209 are changed substantially only by pyrophosphoric acid contained in the sample solution 202. Accordingly, it is possible to accurately estimate the amount of pyrophosphoric acid contained in the sample solution 202.

Alternatively, the membrane 207 may contain protein other than H⁺-pyrophosphatase. However, the protein preferably does not react to or has a low reactivity with pyrophosphoric acid. The reason for this is that when pyrophosphoric acid reacts to the protein other than H⁺-pyrophosphatase in the membrane 207, the amount of pyrophosphoric acid which reacts to H⁺-pyrophosphatase is reduced, resulting in a reduction of the amount of H⁺ to be transported. On the other hand, in the case where the membrane 207 contains protein which does not react to pyrophosphoric acid but reacts to a substance other than phosphoric acid, thereby transporting H⁺, it is preferred that the substance to which the protein reacts is not substantially contained in the sample solution 202. Specifically, when the membrane 207 contains such protein as ATPase which does not react to pyrophosphoric acid but reacts to ATP, thereby transporting H⁺, it is preferred that ATP is not substantially contained in the sample solution 202.

In the pyrophosphoric acid measurement device 200, the amount of pyrophosphoric acid is electrically measured using the electrode 205 and the H⁺-sensitive electrode 208. However, the present invention is not limited to this. For example, a solution containing a pH-sensitive dye or a membrane potential-sensitive dye may be added into the inner region 209 in the inner vessel 206. In this case, the fluorescence intensity of the pH-sensitive dye or the membrane potential-sensitive dye is changed with an increase in concentration of H⁺ within the inner vessel 206. By optically measuring the change of the fluorescence intensity, it is made possible to measure the amount of pyrophosphoric acid.

(Fifth Embodiment)

As in the first through third embodiments, the genomic DNA and the typing primer are used for amplifying a nucleic acid by a base sequence amplification method such as a PCR. Thereafter, the amount of pyrophosphoric acid in a resultant amplification reaction liquid is analyzed by an electrochemical technique using three types of enzymes, i.e., pyrophosphatase, glyceraldehyde 3-phosphatedehyrogenase, and diaphorase, thereby analyzing the amount of amplification of the nucleic acid for determining the base type.

Specifically, in an electrochemical measurement container, firstly, pyrophosphatase is caused to react to a sample solution of the amplification reaction liquid, so as to hydrolyze pyrophosphoric acid obtained by an amplification reaction of the nucleic acid and thereby to generate an inorganic phosphoric acid. The electrochemical measurement container includes a plurality of current measurement electrodes connected to a commercially available electrochemical measurement system (produced by Hokuto Denko Corp., for example).

Next, glyceraldehyde 3-phosphatedehyrogenase is caused to react to the inorganic phosphoric acid under the presence of glyceraldehyde 3-phosphate and oxidized nicotinamide adenine dinucleotide, such that the inorganic phosphoric acid is converted into 1,3-bisphosphoglycerate and reduced nicotinamide adenine dinucleotide.

Then, the resultant reduced nicotinamide adenine dinucleotide is caused to react to diaphorase together with an oxidized form of an electron-transfer mediator (e.g., ferricyanide, 1,2-naphthoquinone-4-sulfonic acid, 2,6-dichlorophenol-indophenol, dimethylbenzoquinone, 1-methoxy-5-methylphenazinium sulfate, methylene blue, gallocyanine, thionine, phenazine methosulfate, or meldora blue), resulting in oxidized nicotinamide adenine dinucleotide and a reduced form of the electron-transfer mediator.

Lastly, on a working electrode in the electrochemical measurement container, the resultant reduced form of the electron-transfer mediator is subjected to electrochemical oxidation, and electrons emitted from the mediator being subjected to the electrochemical oxidation are measured as current values by the electrochemical measurement system. The amount of amplification of the nucleic acid can be measured based on the current values. Based on such quantitative analysis, for example, typing of the SNP site can be achieved, and therefore it is possible to determine the type of a base in the substituted region of the target nucleic acid.

(Sixth Embodiment)

(Kit)

The base type determination primer of the present invention, together with other reagents, etc., required for the primer extension reaction, etc., can be provided as a base type determination reagent kit. Typically, the base type determination reagent kit of the present invention includes the base type determination primer of the present invention, a DNA polymerase, and dNTPs. Preferably, the DNA polymerase has substantially no 3′→5′ exonuclease activity. In the case of using a DNA polymerase having 3′→5′ exonuclease activity, nucleotides in both a substitution corresponding region and an uncomplementary region of the base type determination primer are cut due to the activity of the DNA polymerase even when the substitution corresponding region has a base uncomplementary to a base in a substituted region of a target nucleic acid and therefore is not able to hybridize to the substituted region of the target nucleic acid. Consequently, the primer extension reaction progresses. Accordingly, if the DNA polymerase having substantially no 3′→5′ exonuclease activity is used, it is possible to prevent the nucleotides in the substitution corresponding region and the uncomplementary region from being cut, thereby eliminating a possibility that the type of a base in the substituted region of the target nucleic acid might not be accurately determined.

In the above-mentioned kit, it is preferred that the primer is DNA. DNA is chemically highly stable, and can be readily handled and obtained.

The base type determination reagent kit of the present invention may further include another reagent for use in a base sequence amplification reaction by a PCR or the like. An example of such a reagent is a reverse primer.

Further, the base type determination reagent kit may further include a reagent for measuring the amount of amplified nucleic acid fragments. Pyrophosphatase is a general example of such a reagent. Moreover, in a specific embodiment of the present invention, the base type determination reagent kit may further include glyceraldehyde 3-phosphate, oxidized nicotinamide adenine dinucleotide, glyceraldehyde 3-phosphatedehydrogenase, and an electron-transfer mediator. Preferably, diaphorase may also be included. Examples of the electron-transfer mediator include ferricyanide, 1,2-naphtoquinone-4-sulfonic acid, 2,6-dichlorophenol-indophenol, dimethylbenzoquinone, 1-methoxy-5-methylphenazinium sulfate, methylene blue, gallocyanine, thionine, phenazine methosulfate, and meldora blue. A person of ordinary skill in the art is able to obtain these reagents as commercial products.

In another specific embodiment of the present invention, the base type determination reagent kit may further include H⁺-pyrophosphatase enclosed in a membrane vesicle as another reagent for measuring the amount of amplified DNA fragments. Moreover, it is preferred that the base type determination reagent kit includes any one of a pH test paper, a pH-sensitive dye (e.g., acridine orange), and a membrane potential-sensitive dye (e.g., DiBAC₄ (3) (Bis (1,3-dibutylbarbituric acid) trimethine oxonol), DiBAC₄ (5) (Bis (1,3-dibutylbarbituric acid)pentamethine oxonol), DiSBAC₂ (3) (Bis (1,3-diethylthiobarbituric acid)trimethine oxonol), di-4-ANNEPS, DiOC₆ (3) (dihexaoxacarbocyanine iodide), or oxonol V).

In a preferred embodiment of the present invention, the base type determination reagent kit may further include a second base type determination primer. The second base type determination primer consists of a second single-stranded nucleic acid capable of hybridizing to the target nucleic acid such that the 3′ terminal of the primer corresponds to the substituted region of the same strand as that to which the first base type determination primer may hybridize. The second single-stranded nucleic acid includes: a second substitution corresponding region located at the 3′ terminal and consisting of a base which is complementary to any one of predictable types of bases in the substituted region of the target nucleic acid and is different in type from a base in the substitution corresponding region of a first single-stranded nucleic acid; a second uncomplementary region which is adjacent to the second substitution corresponding region on the 5′ terminal side thereof and consists of at least two bases uncomplementary to the target nucleic acid; and a second complementary region which is adjacent to the second uncomplementary region on the 5′ terminal side thereof and complementary to the target nucleic acid. The second complementary region consists of a sufficient number of bases to hybridize to the target nucleic acid under such conditions that the primer extension reaction of the second single-stranded nucleic acid can occur at least when a base in the substituted region of the target nucleic acid is complementary to a base in the second substitution corresponding region. Most preferably, the second substitution corresponding region consists of a single base, the second uncomplementary region consists of two bases, and the second complementary region consists of five or more bases.

It is preferred that the first single-stranded nucleic acid and the second single-stranded nucleic acids are different in length from each other. The reason for this is that it can be readily determined whether an amplified base sequence is derived from the first or second base type determination primer. For a reason similar to this, it is also preferred that the first single-stranded nucleic acid and the second single-stranded nucleic acid are labeled by their respective fluorescences which are different in wavelength. Examples of label reagents with different wavelengths include a combination of Cy3™ (Amersham Life Science Inc.) and Cy5™ (Amersham Life Science Inc.) and any combination of label reagents selected from the group consisting of JOE (having an emission peak at 550 nm), ROX (having an emission peak at 600 nm), FAM (having an emission peak at 520 nm) and TAMRA (having an emission peak at 580 nm). The above label reagents can be readily obtained as commercial products from suppliers, such as Invitrogen Corp. and Funakoshi Co. Ltd., which are well-known to those skilled in the art.

Preferably, the base type determination reagent kit of the present invention may further include instructions specifying directions and procedures for use of reagents and other information about precautions.

### EXAMPLE 1

In Example 1, a solution of genomic DNAs extracted from human blood was used to attempt to type an SNP existing in an ADH2 gene. In the attached SEQUENCE LISTING, SEQ. ID No. 10 indicates a base sequence which includes an SNP site targeted for analysis in the present example and genomic DNAs in the vicinity of the SNP site. The SNP site is the forty-sixth base (a site denoted by r) in the sequence indicated by SEQ. ID No. 10, and might be A or G. In this case, there are three possible SNP patterns, i.e., A/A homo, G/G homo, and A/G hetero.

Firstly, GEN [symbol] ™ (for use with blood) (Takara Shuzo Co., Ltd.) was used to extract genomic DNA of an SNP pattern of A/A (hereinafter, referred to as an “A/A genomic DNA”), genomic DNA of an SNP pattern of A/G (hereinafter, referred to as an “A/G genomic DNA”), and genomic DNA of an SNP pattern of G/G (hereinafter, referred to as a “G/G genomic DNA”) from bloods of three subjects whose SNP patterns are previously known to be A/A, A/G, and G/G, respectively.

Next, two types of typing primers identified by SEQ. ID Nos. 11 and 12 and a reverse primer identified by SEQ. ID No. 13 were used to prepare PCR reaction liquids 1 through 6, and a PCR reaction was carried out. Compositions of each PCR reaction liquid are as shown in Tables 2 through 7 below. Conditions of the PCR reaction are as shown in FIG. 11. Here, the number of repetitions is 20.

TABLE 2
PCR reaction liquid 1

| Contents (concentration) | Volume |
|---|---|
| A/A genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Typing primer 1 of SEQ. ID No. 11 (20 μM) | 0.9 μl |
| Reverse primer of SEQ. ID No. 13 (20 μM) | 0.9 μl |
| Distilled water | 13.5 μl |

TABLE 3
PCR reaction liquid 2

| Contents (concentration) | Volume |
|---|---|
| A/A genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Typing primer of SEQ. ID No. 12 (20 μM) | 0.9 μl |
| Reverse primer of SEQ. ID No. 13 (20 μM) | 0.9 μl |
| Distilled water | 13.5 μl |

TABLE 4
PCR reaction liquid 3

| Contents (concentration) | Volume |
|---|---|
| A/G genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Typing primer of SEQ. ID No. 11 (20 μM) | 0.9 μl |
| Reverse primer of SEQ. ID No. 13 (20 μM) | 0.9 μl |
| Distilled water | 13.5 μl |

TABLE 5
PCR reaction liquid 4

| Contents (concentration) | Volume |
|---|---|
| A/G genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Typing primer of SEQ. ID No. 12 (20 μM) | 0.9 μl |
| Reverse primer of SEQ. ID No. 13 (20 μM) | 0.9 μl |
| Distilled water | 13.5 μl |

TABLE 6
PCR reaction liquid 5

| Contents (concentration) | Volume |
|---|---|
| G/G genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Typing primer of SEQ. ID No. 11 (20 μM) | 0.9 μl |
| Reverse primer of SEQ. ID No. 13 (20 μM) | 0.9 μl |
| Distilled water | 13.5 μl |

TABLE 7
PCR reaction liquid 6

| Contents (concentration) | Volume |
|---|---|
| G/G genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Typing primer of SEQ. ID No. 12 (20 μM) | 0.9 μl |
| Reverse primer of SEQ. ID No. 13 (20 μM) | 0.9 μl |
| Distilled water | 13.5 μl |

After the PCR reaction, electrophoresis was carried out using a 3% agarose gel.

Amplification of a target DNA fragment was observed in the PCR reaction liquids 1, 3, 4, and 6. On the other hand, almost no amplification was observed in the PCR reaction liquids 2 and 5. That is, regarding the A/A genomic DNA, the amplification was observed only when the typing primer of SEQ. ID No. 11 was used. In contrast, as for the G/G genomic DNA, the amplification was observed only when the typing primer of SEQ. ID No. 12 was used. As for the A/G genomic DNA, the amplification was observed when either of the typing primers was used.

Consequently, it was confirmed that a base in the SNP site can be determined using the typing primers of SEQ. ID Nos. 11 and 12.

Regarding the amount of amplification of the DNA fragment, substantially the same amount was observed in the PCR reaction liquids 1 and 6, and the amount observed in each of the PCR reaction liquids 3 and 4 was apparently lower than the amount observed in each of the PCR reaction liquids 1 and 6. In this manner, there is confirmed a clear difference in the amount of amplification of the DNA fragment between the reaction liquids 1 and 3, although the same typing primer was used. This is because as compared to the PCR reaction liquid 3, the PCR reaction liquid 1 has twice the amount of genomic DNA having A as a base at the SNP site which is used as a template in a PCR reaction.

The above results show that it is also possible to analyze whether the SNP pattern of a genomic DNA is homo or hetero based on a difference in the amount of amplification of the DNA fragment. Accordingly, in the present example, even if only either one of the typing primers of SEQ. ID Nos. 11 and 12 is used, it is possible to type three kinds of SNP patterns of A/A, A/G and G/G by analyzing not only the presence or absence of amplification of the DNA fragment but also the amount of amplification of the DNA fragment. Such a quantitative SNP pattern analysis based on the amount of amplification of the DNA fragment is made possible only by the typing primer of the present invention which provides a clear distinction between a case where the primer extension reaction progresses and a case where the primer extension reaction does not progress.

The above results show that SNP typing can be achieved by using the typing primer of the present invention.

### EXAMPLE 2

In the present example, the same genomic DNA as used in Example 1 was used as a target for analysis, and two typing primers of different lengths were used to attempt to provide SNP typing. The same SNP site as analyzed in Example 1 was analyzed.

In the present example, a primer solution (20 μM) was prepared for each of the following primers: a typing primer obtained by the 5′ terminal of the typing primer 1 of SEQ. ID No. 11 used in Example 1 with 6FAM (hereinafter, referred to as a “labeled primer C”); a typing primer obtained by labeling the 5′ terminal of oligonucleotide indicated by SEQ. ID No. 14 with 6FAM (hereinafter, referred to as a “labeled primer D”); and a reverse primer identified by SEQ. ID No. 15.

Here, the total length of an amplified DNA fragment obtained by a PCR from both the labeled primer C and the reverse primer identified by SEQ. ID No. 15 corresponds to 60 base pairs (bp), while the total length of an amplified DNA fragment obtained by a PCR from both the labeled primer D and the reverse primer identified by SEQ. ID No. 15 corresponds to 62 bp.

Next, the labeled primers C and D and the reverse primer identified by SEQ. ID No. 15 were used to prepare PCR reaction liquids 7 though 9, and a PCR reaction was provided in each prepared PCR reaction liquid. Compositions of each PCR reaction liquid are as shown in Tables 8 through 10 below. As in the case of Example 1, conditions of the PCR reaction are as shown in FIG. 11. Here, the number of repetitions is 20.

TABLE 8
PCR reaction liquid 7

| Contents (concentration) | Volume |
|---|---|
| A/A genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Labeled primer C (20 μM) | 0.9 μl |
| Labeled primer D (20 μM) | 0.9 μl |
| Reverse primer of SEQ. ID No. 15 (20 μM) | 0.9 μl |
| Distilled water | 12.6 μl |

TABLE 9
PCR reaction liquid 8

| Contents (concentration) | Volume |
|---|---|
| A/G genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Labeled primer C (20 μM) | 0.9 μl |
| Labeled primer D (20 μM) | 0.9 μl |
| Reverse primer of SEQ. ID No. 15 (20 μM) | 0.9 μl |
| Distilled water | 12.6 μl |

TABLE 10
PCR reaction liquid 9

| Contents (concentration) | Volume |
|---|---|
| G/G genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Labeled primer C (20 μM) | 0.9 μl |
| Labeled primer D (20 μM) | 0.9 μl |
| Reverse primer of SEQ. ID No. 15 (20 μM) | 0.9 μl |
| Distilled water | 12.6 μl |

After the PCR reaction, each PCR reaction liquid was analyzed using a genetic analyzer ABIPRISM310 (produced by Applied Biosystems Japan Ltd.).

As a result, regarding the PCR reaction liquid 7, a peak indicating that a 60-bp DNA fragment was predominantly amplified was observed, while no peak for indicating amplification of a 62-bp DNA fragment was observed. In contrast, as for the PCR reaction liquid 9, a peak indicating amplification of the 62-bp DNA fragment was observed, while almost no peak for indicating amplification of the 60-bp DNA fragment was observed. As for the PCR reaction liquid 8, a peak indicating amplification of each of the 60- and 62-bp base sequences was observed.

Consequently, it was confirmed that by using the labeled primer C and D, it is possible to accurately type the three kinds of SNP patterns of A/A, A/G and G/G.

### EXAMPLE 3

In the present example, after a PCR reaction was carried out using the same genomic DNA and typing primers as those used in Example 1, typing of a base in the SNP site of the genomic DNA was attempted by analyzing the amount of pyrophosphoric acid contained in a resultant PCR reaction liquid using a luciferase reaction.

Similar to Example 1, firstly, the PCR reaction liquids 1 through 6 were prepared and a PCR reaction was carried out.

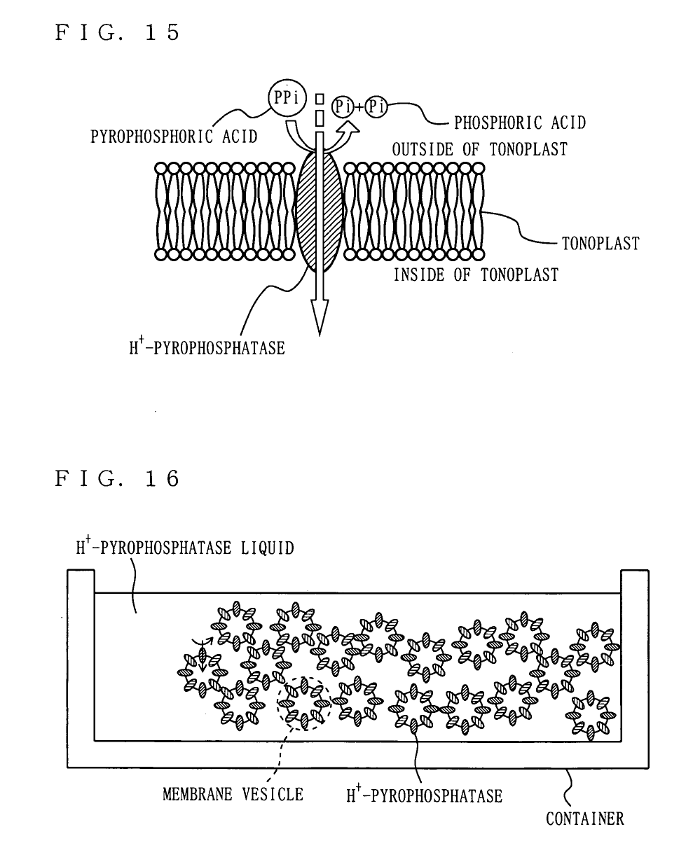

Next, the amount of pyrophosphoric acid contained in each PCR reaction liquid subjected to the PCR reaction was analyzed in accordance with a method by Mostafa Ronaghi et al. (Ronaghi, M., Uhlen, M. and Nyren, P. (1998) “A sequencing method based on real-time pyrophosphate”, Science, 281, 363-365). Analysis of luminescence intensity by a luciferase reaction was carried out using an AQUACOSMOS/VIM system (produced by Hamamatsu Photonics K.K.). The result of analysis is shown in FIG. 12.

In FIG. 12, the luminescence intensity in the PCR reaction liquid 1 is taken as 100%, and each luminescence intensity in other PCR reaction liquids is represented by a percentage relative to the PCR reaction liquid 1 (=(the luminescence intensity in each PCR reaction liquid/the luminescence intensity in the PCR reaction liquid 1)×100).

As is apparent from FIG. 12, it was observed that luminescence intensities in the PCR reaction liquids 1 and 6 are almost equivalent to each other. Although it was observed that luminescence intensities in the PCR reaction liquids 3 and 4 are almost equivalent to each other, their luminescence intensities are apparently lower than the luminescence intensities in the PCR reaction liquids 1 and 6. As for the PCR reaction liquids 2 and 5, almost no luminescence was observed.

Accordingly, regarding the A/A genomic DNA, luminescence was observed only when the typing primer of SEQ. ID No. 11 was used. In contrast, as for the G/G genomic DNA, luminescence was observed only when the typing primer of SEQ. ID No. 12 was used. As for the A/G genomic DNA, although luminescence was observed when either of the typing primers of SEQ. ID Nos. 11 and 12 was used, the luminescence intensity in this case was apparently lower than that in the above two cases.

In the present example, typing of the SNP site targeted for analysis was achieved by using the above-described typing primers to analyze the presence or absence of the luminescence by means of the PCR reaction and the luciferase reaction.

Further, as described above, regarding the A/G genomic DNA, the luminescence intensity in the case of using the typing primers of SEQ. ID Nos. 11 and 12 was apparently lower than that in the case of using the typing primer of SEQ. ID No. 11 for the A/A genomic DNA or in the case of using the typing primer of SEQ. ID No. 12 for the G/G genomic DNA. The reason for this is that in each of the A/A genomic DNA and the G/G genomic DNA, the amount of genomic DNA having A or G as a base at the SNP site, which is used as a template in the PCR reaction, is twice as much as in the A/G genomic DNA.

Consequently, it was confirmed that not only by analyzing the presence or absence of the luminescence but also by carrying out a quantitative analysis of the luminescence intensity, it is made possible to provide SNP typing even when only one of the typing primers of SEQ. ID Nos. 11 and 12 is used. Such a quantitative analysis-based determination as to whether the SNP pattern is homo or hetero can be advantageously implemented only by the present invention which provides a clear distinction between a case where the primer extension reaction progresses and a case where the primer extension reaction does not progress.

### Example 4

In the present example, a solution of genomic DNAs extracted from human blood was used to attempt to type an SNP existing in a carbohydrate sulfo transferase 2 gene. A base sequence, which includes an SNP site targeted for analysis in the present example and genomic DNAs in the vicinity of the SNP site, is indicated by SEQ. ID No. 16. The SNP site is the thirty-seventh base (a site denoted by s) in the sequence indicated by SEQ. ID No. 16, and might be G or C.

Firstly, GEN [symbol] ™ (for use with blood) (Takara Shuzo Co. Ltd.) was used to extract genomic DNA of an SNP pattern of G/G (hereinafter, referred to as a “G/G genomic DNA”), genomic DNA of an SNP pattern of G/C (hereinafter, referred to as a “G/C genomic DNA”), and genomic DNA of an SNP pattern of C/C (hereinafter, referred to as a “C/C genomic DNA”) from bloods of three subjects whose SNP patterns are previously known to be G/G, G/C, and C/C, respectively.

Next, a primer identified by SEQ. ID No. 17 and a reverse primer identified by SEQ. ID No. 18 were used to prepare PCR reaction liquids 10 through 12, and a PCR reaction was carried out. Compositions of each PCR reaction liquid are as shown in Tables 11 through 13 below. Conditions of the PCR reaction are as shown in FIG. 11. Here, the number of repetitions is 35.

TABLE 11
PCR reaction liquid 10

| Contents (concentration) | Volume |
|---|---|
| G/G genomic DNA (100 ng/μl) | 1 μl |
| Takara Taq ™ (5 U/μl) | 0.1 μl |
| 10 × PCR buffer | 2 μl |
| dNTPs (2.5 mM) | 1.6 μl |
| Primer of SEQ. ID NO. 17 (20 μM) | 0.9 μl |
| Reverse primer of SEQ. ID NO. 18 (20 μM) | 0.9 μl |
| Distilled water | 13.5 μl |

TABLE 12
PCR reaction liquid 11

| Contents (concentration) | Volume |
|---|---|
| G/C genomic DNA (100 ng/μl) | 1 μl |
| Takara Taq ™ (5 U/μl) | 0.1 μl |
| 10 × PCR buffer | 2 μl |
| dNTPs (2.5 mM) | 1.6 μl |
| Primer of SEQ. ID NO. 17 (20 μM) | 0.9 μl |
| Reverse primer of SEQ. ID NO. 18 (20 μM) | 0.9 μl |
| Distilled water | 13.5 μl |

TABLE 13
PCR reaction liquid 12

| Contents (concentration) | Volume |
|---|---|
| C/C genomic DNA (100 ng/μl) | 1 μl |
| Takara Taq ™ (5 U/μl) | 0.1 μl |
| 10 × PCR buffer | 2 μl |
| dNTPs (2.5 mM) | 1.6 μl |
| Primer of SEQ. ID NO. 17 (20 μM) | 0.9 μl |
| Reverse primer of SEQ. ID NO. 18 (20 μM) | 0.9 μl |
| Distilled water | 13.5 μl |

After the PCR reaction, each of the PCR reaction liquids 10 through 12 was purified using a SUPREC™-PCR (Takara Shuzo, Co., Ltd.). Typing primers identified by SEQ. ID Nos. 19 and 20 were added to the purified PCR reaction liquids to prepare primer extension reaction liquids 1 through 6, and a primer extension reaction was carried out. Compositions of each primer extension reaction liquid are as shown in Tables 14 through 19 below. Conditions of the primer extension reaction is as shown in FIG. 13.

TABLE 14
Primer extension reaction liquid 1

| Contents (concentration) | Volume |
|---|---|
| Purified PCR reaction liquid 10 | 10 μl |
| Platinum Taq DNA polymerase (5 U/μl) | 0.1 μl |
| 10 × PCR buffer, Minus Mg | 2 μl |
| MgCl₂ (50 mM) | 0.8 μl |
| dNTPs (2.5 mM) | 1.6 μl |
| Typing primer of SEQ. ID No. 19 (20 μm) | 0.9 μl |
| Distilled water | 4.6 μl |

TABLE 15
Primer extension reaction liquid 2

| Contents (concentration) | Volume |
|---|---|
| Purified PCR reaction liquid 10 | 10 μl |
| Platinum Taq DNA polymerase (5 U/μl) | 0.1 μl |
| 10 × PCR buffer, Minus Mg | 2 μl |
| MgCl₂ (50 mM) | 0.8 μl |
| dNTPs (2.5 mM) | 1.6 μl |
| Typing primer of SEQ. ID No. 20 (20 μm) | 0.9 μl |
| Distilled water | 4.6 μl |

TABLE 16
Primer extension reaction liquid 3

| Contents (concentration) | Volume |
|---|---|
| Purified PCR reaction liquid 11 | 10 μl |
| Platinum Taq DNA polymerase (5 U/μl) | 0.1 μl |
| 10 × PCR buffer, Minus Mg | 2 μl |
| MgCl₂ (50 mM) | 0.8 μl |
| dNTPs (2.5 mM) | 1.6 μl |
| Typing primer of SEQ. ID No. 19 (20 μm) | 0.9 μl |
| Distilled water | 4.6 μl |

TABLE 17
Primer extension reaction liquid 4

| Contents (concentration) | Volume |
|---|---|
| Purified PCR reaction liquid 11 | 10 μl |
| Platinum Taq DNA polymerase (5 U/μl) | 0.1 μl |
| 10 × PCR buffer, Minus Mg | 2 μl |
| MgCl₂ (50 mM) | 0.8 μl |
| dNTPs (2.5 mM) | 1.6 μl |
| Typing primer of SEQ. ID No. 20 (20 μm) | 0.9 μl |
| Distilled water | 4.6 μl |

TABLE 18
Primer extension reaction liquid 5

| Contents (concentration) | Volume |
|---|---|
| Purified PCR reaction liquid 12 | 10 μl |
| Platinum Taq DNA polymerase (5 U/μl) | 0.1 μl |
| 10 × PCR buffer, Minus Mg | 2 μl |
| MgCl₂ (50 mM) | 0.8 μl |
| dNTPs (2.5 mM) | 1.6 μl |
| Typing primer of SEQ. ID No. 19 (20 μm) | 0.9 μl |
| Distilled water | 4.6 μl |

TABLE 19
Primer extension reaction liquid 6

| Contents (concentration) | Volume |
|---|---|
| Purified PCR reaction liquid 12 | 10 μl |
| Platinum Taq DNA polymerase (5 U/μl) | 0.1 μl |
| 10 × PCR buffer, Minus Mg | 2 μl |
| MgCl₂ (50 mM) | 0.8 μl |
| dNTPs (2.5 mM) | 1.6 μl |
| Typing primer of SEQ. ID No. 20 (20 μm) | 0.9 μl |
| Distilled water | 4.6 μl |

After the primer extension reaction, the amount of pyrophosphoric acid contained in each primer extension reaction liquid was analyzed by means of a luciferase reaction in the same manner as in Example 3. The result for analysis is shown in FIG. 14.

In FIG. 14, the luminescence intensity in the primer reaction liquid 1 is taken as 100%, and each luminescence intensity in other primer extension reaction liquids is represented by a percentage relative to the primer extension reaction liquid 1 (=(the luminescence intensity in each primer extension reaction liquid/the luminescence intensity in the primer extension reaction liquid 1)×100).

As is apparent from FIG. 14, it was observed that luminescence intensities in the primer extension reaction liquids 1 and 6 are almost equivalent to each other. Although it was observed that luminescence intensities in the primer extension reaction liquids 3 and 4 are almost equivalent to each other, their luminescence intensities are apparently lower than the luminescence intensities in the primer extension reaction liquids 1 and 6. As for the primer extension reaction liquids 2 and 5, almost no luminescence was observed.

Accordingly, regarding the G/G genomic DNA, luminescence was observed only when the typing primer of SEQ. ID No. 19 was used. In contrast, as for the C/C genomic DNA, luminescence was observed only when the typing primer of SEQ. ID No. 20 was used. As for the G/C genomic DNA, although luminescence was observed when either of the typing primers of SEQ. ID Nos. 19 and 20 was used, the luminescence intensity in this case was apparently lower than that in the above two cases.

In the present example, typing of the SNP site targeted for analysis was achieved by using the above-described typing primers to analyze the presence or absence of the luminescence by means of the primer extension reaction and the luciferase reaction.

Further, as described above, regarding the G/C genomic DNA, the luminescence intensity in the case of using the typing primers of SEQ. ID Nos. 19 and 20 was apparently lower than that in the case of using the typing primer of SEQ. ID No. 19 for the G/G genomic DNA or in the case of using the typing primer of SEQ. ID No. 20 for the C/C genomic DNA. The reason for this is that in each of the G/G genomic DNA and the C/C genomic DNA, the amount of genomic DNA having G or C as a base at the SNP site, which is used as a template in the primer extension reaction, is twice as much as in the G/C genomic DNA.

Consequently, it was confirmed that not only by analyzing the presence or absence of the luminescence but also by carrying out a quantitative analysis of the luminescence intensity, it is made possible to provide SNP typing even when only one of the typing primers of SEQ. ID Nos. 19 and 20 is used. Such a quantitative analysis-based determination as to whether the SNP pattern is homo or hetero can be advantageously implemented only by the present invention which provides a clear distinction between a case where the primer extension reaction progresses and a case where the primer extension reaction does not progress.

### EXAMPLE 5

In Example 5, a PCR reaction was carried out using the same genomic DNAs and primers as those used in Example 1, and thereafter pyrophosphoric acid in a resultant PCR reaction liquid was analyzed using H⁺-pyrophosphatase to attempt to type the same SNP site as that targeted for analysis in Example 1.

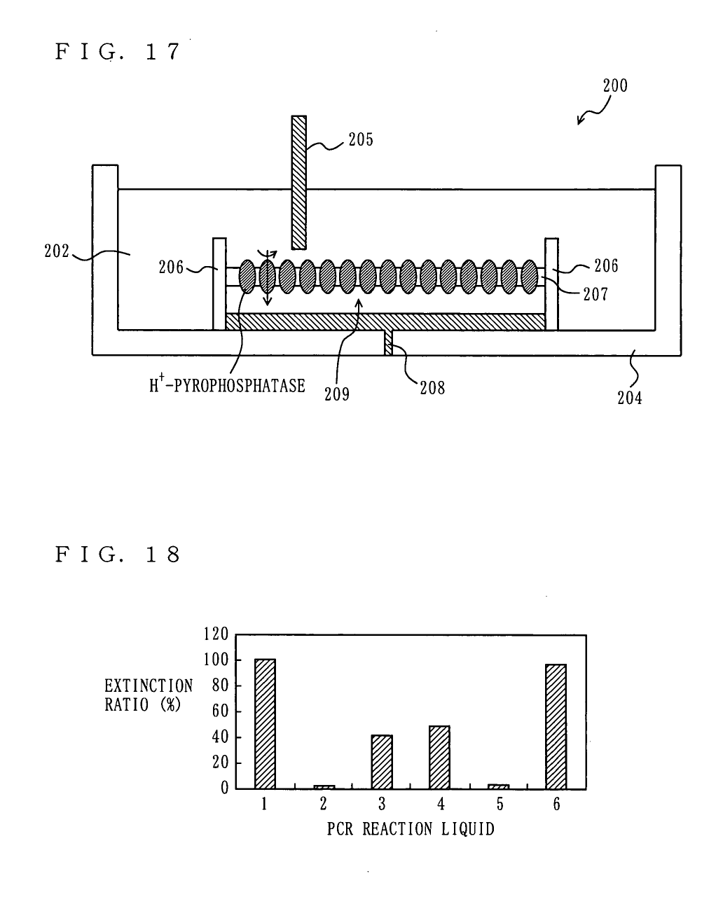

FIG. 15 is a diagram schematically illustrating H⁺-pyrophosphatase.

In general, H⁺-phosphatase is a membrane protein present within a tonoplast of a plant, and has, as can be seen from FIG. 15, a property of transporting H⁺ from the outside of the tonoplast into the inside of the tonoplast, while providing a hydrolysis reaction which generates a bimolecular phosphoric acid from a monomolecular pyrophosphoric acid. Therefore, due to an enzyme reaction of H⁺-pyrophosphatase, the concentration of H⁺ is increased in the inside of the tonoplast and decreased in the outside thereof. Accordingly, in order to measure pyrophosphoric acid, a sample liquid containing pyrophosphoric acid to be measured may be made to be in contact with H⁺-pyrophosphatase present within a tonoplast isolated from a plant cell or the like, and thereafter a change in concentration of H⁺ in the inside or outside of the tonoplast may be measured. In this case, H⁺-pyrophosphatase is not necessarily used in the state of being bound to the tonoplast isolated from a cell. For example, after having been isolated from the tonoplast, H⁺-pyrophosphatase may be reconstructured in a membrane, such as an artificially formed lipid bilayer film, which transmits substantially no H⁺therethrough. Note that it is a common practice of a person of ordinary skill in the art to extract and/or produce a natural or artificial tonoplast enclosing H⁺-pyrophosphatase therein.

Typical examples of a method for measuring a change in concentration of H⁺ include a method which converts the change in concentration of H⁺ into an optical or electrical change and measures the optical or electrical change. Examples of a method, which converts the change in concentration of H⁺ into an optical change and measures the optical change, includes methods which use a pH test paper, a pH-sensitive dye (e.g., acridine orange), or a membrane potential-sensitive dye (e.g., DiBAC₄ (3) (Bis (1,3-dibutylbarbituric acid)trimethine oxonol), DiBAC₄ (5) (Bis (1,3-dibutylbarbituric acid)pentamethine oxonol), DiSBAC₂ (3) (Bis (1,3-diethylthiobarbituric acid)trimethine oxonol), di-4-ANNEPS, DiOC₆ (3) (dihexaoxacarbocyanine iodide), or oxonol V). Examples of the method which converts the change in concentration of H⁺ into an electrical change and measures the electrical change include a metal-electrode method (e.g., a hydrogen-electrode method, a quinhydrone-electrode method, or an antymony-electrode method), a glass-electrode method, an ion-selective field-effective transistor electrode (ISFET) method, a patch-clamp method, or a light-addressable potentiometric sensor (LAPS) method. By using such a method of measuring a change in concentration of H⁺ together with the above-described reaction of H⁺-pyrophosphatase, pyrophosphoric acid in a sample liquid can be measured after having been converted into an optical or electric signal. Note that in the present invention, a method used for analyzing a change in concentration of H⁺ is not limited to the above-described method for measuring a change in concentration of H⁺. Any analysis method can be used so long as the change in concentration of H⁺ can be analyzed.

In the present example, an H⁺-pyrophosphatase liquid containing a tonoplast derived from a green gram was prepared in the following manner conforming to a method of Shizuo Yoshida et al. (Masayoshi Maeshima and Shizuo Yoshida, (1989), J. Biol. Chem. 264(33), pp. 20068 to 20073).

Firstly, an endoplasmic reticulum of a green gram-derived tonoplast was dissolved in a solution consisting of Tris/Mes (concentration: 5 mM, pH: 7.0), sorbitol (concentration: 0.25 M), and DTT (concentration: 2 mM), resulting in a membrane vesicle suspension liquid of a tonoplast.

Next, the suspension liquid was mixed with a reaction liquid consisting of MgSO₄ (concentration: 1 mM), KCl (concentration: 50 mM), sorbitol (concentration: 0.25 M), acridine orange (i.e., a ph-sensitive dye, 3 μM), and Hepes/Bristris propane (concentration: 25 mM, pH: 7.2), resulting in an H⁺-pyrophosphatase liquid. FIG. 16 schematically illustrates the mixture of the suspension liquid with the reaction liquid. Here, each membrane vesicle holds H⁺-pyrophosphatase, and acridine orange is uniformly present in the inside and outside of the membrane vesicle. The H⁺-pyrophosphatase liquid was equally separated and injected into six tubes.

Next, as in Example 1, each of the PCR reaction liquids 1 through 6 subjected to a PCR reaction was separately added to a tube containing the above-described H⁺-pyrophosphatase liquid, and a reaction was caused to occur due to H⁺-pyrophosphatase.

In the present example, acridine orange is used as a pH-sensitive dye. Acridine orange can be transmitted through a tonoplast, and has a property of quenching its fluorescence under acidic conditions. Accordingly, when adding to the H⁺-pyrophosphatase liquid a solution containing a prescribed amount or more than the prescribed amount of pyrophosphoric acid, H⁺ transport is caused by H⁺-pyrophosphatase, thereby acidifying the inside of each membrane vesicle. Therefore, the fluorescence of acridine orange is quenched. By exploiting such a property of acridine orange, it is made possible to quantify the amount of pyrophosphoric acid produced by the PCR reaction.

In the present example, a change of the fluorescent intensity (excitation light: 493 nm, fluorescence: 540 nm) of acridine orange was analyzed before and after each PCR reaction was added. The result for analysis is shown in FIG. 18.

FIG. 18 is a graph showing a change of a fluorescence intensity of 540 nm for each of the above-described PCR reaction liquids. The change of the fluorescence of 540 nm is represented by an extinction ratio per unit of second at one second after addition of each PCR reaction liquid. In FIG. 10, an extinction ratio per unit of second for each PCR reaction liquid is provided on the premise that an extinction ratio per unit of second in the case of the PCR reaction liquid 1 is 100%.

From FIG. 18, it is appreciated that extinction ratios of the PCR reaction liquids 1 and 6 are almost equivalent to each other. Although extinction ratios of the PCR reaction liquids 3 and 4 are almost equivalent to each other, their extinction ratios are apparently lower than those of the PCR reaction liquids 1 and 6. As for the PCR reaction liquids 2 and 5, there is substantially no quenching of fluorescence.

Consequently, regarding the A/A genomic DNA, there is quenching of fluorescence only when the typing primer identified by SEQ. ID No. 11 is used. In contrast, as for the G/G genomic DNA, there is quenching of fluorescence only when the typing primer identified by SEQ. ID No. 12 is used. As for the A/G genomic DNA, although there is quenching of fluorescence when either of the typing primers identified by SEQ. ID Nos. 11 and 12 is used, their extinction ratios are apparently lower than that in the above two cases.

Accordingly, in the present example, the typing primers identified by SEQ. ID Nos. 11 and 12 were used to provide a PCR reaction, and the extinction ratios were analyzed using a reaction due to H⁺-pyrophosphatase, thereby typing the SNP site. As described above, the extinction ratio of the A/G genomic DNA measured with use of the typing primer identified by SEQ. ID No. 11 or 12 is apparently lower than the extinction ratio of the A/A genomic DNA measured with use of the typing primer identified by SEQ. ID No. 11 and the extinction ratio of the G/G genomic DNA measured with use of the typing primer identified by SEQ. ID No. 12. The reason for this is that in each of the A/A genomic DNA and G/G genomic DNA, the amount of genomic DNA having A or G as a base at the SNP site which is used as a template in a PCR reaction is twice as much as in the A/G genomic DNA.

Therefore, by conducting not only analysis of the presence or absence of quenching of fluorescence but also quantitative analysis of the extinction ratio, it is made possible to achieve SNP typing even when only one of the typing primers identified by SEQ. ID Nos. 11 and 12 is used. Such a quantitative analysis-based determination as to whether the SNP pattern is homo or hetero can be advantageously implemented only by the present invention which provides a clear distinction between a case where the primer extension reaction progresses and a case where the primer extension reaction does not progress.

### EXAMPLE 6

In Example 6, a PCR reaction was carried out using the same genomic DNA and primers as those used in Example 1, and thereafter pyrophosphoric acid in a resultant PCR reaction liquid was analyzed using three types of enzymes, i.e., pyrophosphatase, glyceraldehyde-3-phosphate dehydrogenase, and diaphorase, thereby attempting to type the same SNP site as that targeted for analysis in Example 1.

In this method, firstly, pyrophosphatase, which is an enzyme serving as a catalyst for a reaction with pyrophosphoric acid, is used to hydrolyze pyrophosphoric acid, thereby generating an inorganic phosphoric acid.

Next, the inorganic phosphoric acid, as well as glyceraldehyde-3-phosphate and oxidized nicotinamide adenine dinucleotide, are subjected to a catalysis of glyceraldehyde-3-phosphate dehydrogenase, resulting in 1,3-bisphosphoglyceric acid and reduced nicotinamide adenine dinucleotide.

Then, the resultant reduced nicotinamide adenine dinucleotide is subjected to a catalysis of diaphorase, and oxidized by reacting to diaphorase together with oxidized forms of electron-transfer mediators (e.g., ferricyanide, 1,2-naphtoquinone-4-sulfonic acid, 2,6-dichlorophenol-indophenol, dimethylbenzoquinone, 1-methoxy-5-methylphenazinium sulfate, methylene blue, gallocyanine, thionine, phenazine methosulfate, or meldora blue), resulting back in oxidized nicotinamide adenine dinucleotide. In this case, the oxidized forms of the electron-transfer mediators are reduced to become reduced forms. These electron-transfer mediators are well-known to those skilled in the art, and can be readily obtained as commercial products.

Lastly, the resultant reductants of the electron-transfer mediators are electrochemically oxidized on a working electrode to which a prescribed potential or more than the prescribed potential is applied. This oxidation causes emission of electrons, and such emission of electrons can be represented by a value of a current. Such a current can be measured using an ordinary electrochemical detector. Potassium ferricyanide or the like can be preferably used as an electron-transfer mediator.

A value of an oxidation current depends on the amount of pyrophosphoric acid at the beginning of reaction, and pyrophosphoric acid can be detected based on the oxidization current value.

The detection method as described above makes it possible to electrochemically detect pyrophosphoric acid. A value of current obtained as a result of the detection also depends on the amount of pyrophosphoric acid at the beginning of reaction, and therefore the detection is carried out by a quantitative approach.

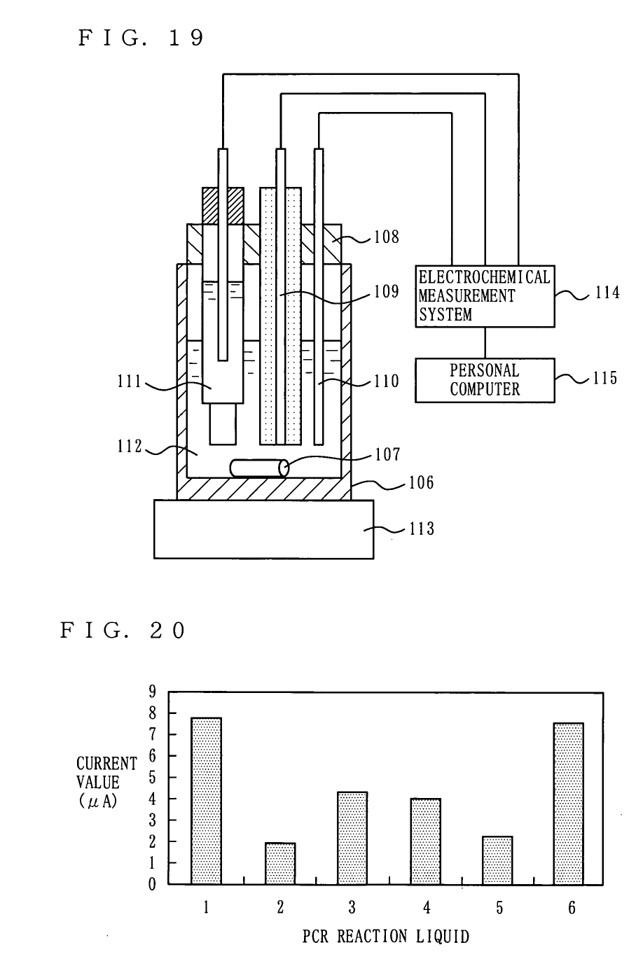

In the present example, firstly, a reaction solution was prepared in the following procedure. 17.5 μl of 30 mM glyceraldehyde 3-phosphoric acid (final concentration: 1 mM), 50 μl of 10 mM (oxidized) nicotinamide adenine dinucleotide (final concentration: 1 mM), 50 μl of 10 mM potassium ferricyanide (final concentration: 1 mM), 8 μl of 100 mM magnesium chloride (final concentration: 1.6 mM), 5 μl of 100 unit/ml diaphorase (final concentration: 10 unit/ml), and 1 μl of 200 unit/ml pyrophosphatase (final concentration: 0.4 unit/ml) were dissolved in a 50 mM Tricine-NaOH buffer solution, and a resultant solution was adjusted to pH 8.8 and amounts to 400 μl. As in Example 1, the resultant solution was added with 50 μl of each of the PCR reaction liquids 1 through 6 subjected to a PCR reaction, thereby producing the reaction solution. The thus-produced reaction solution was introduced into a measurement device system as shown in FIG. 19. The measurement device system was configured as follows. A stirring bar 107 was placed in a glass cell 106, and the glass cell 106 was fixed on a stirrer machine 113. An electrode fixing device 108 was used to set a measurement electrode 109, a counter electrode 110, and a reference electrode 111 within the glass cell 106. The measurement electrode 109 and the counter electrode 110 are formed by a gold electrode and a platinum electrode, respectively, and each of them is 1.6 mm in diameter. The reference electrode 111 is formed by a silver/silver chloride electrode. A porous glass containing a silver chloride-coated silver wire and a saturated KCl solution was connected to the glass cell. Each electrode was connected to an electrochemical measurement system (produced by Hokuto Denko Corp. which is denoted by reference numeral 114 in the figure), and a personal computer 115 was operated to control the system and perform data recording. The glass cell 106 was filled with the above-described reaction solution. The reaction solution was stirred by the stirring bar. The electrochemical measurement system was operated so as to apply to the measurement electrode 109 a voltage of +600 mV over the reference electrode 11. Measurement of voltage was started simultaneously with voltage application, and thereafter a reaction was started by adding 20 μl of 800 unit/ml glyceraldehyde-3-phosphate dehydrogenase (final concentration: 32 unit/ml) to the reaction solution. As a result, mediators in the reaction solution were oxidized, and the oxidation caused emission of electrons. A value of current applied between the measurement electrode 109 and the counter electrode 110 due to the electron emission was measured using the electrochemical measurement system. Current values at sixty seconds after measurement are shown in FIG. 20.

From FIG. 20, it is appreciated that current values of the PCR reaction liquids 1 and 6 are almost equivalent to each other. Although current values of the PCR reaction liquids 3 and 4 are almost equivalent to each other, their current values are apparently lower than those of the PCR reaction liquids 1 and 6. Current values of the PCR reaction liquids 2 and 5 are further lower.

Consequently, regarding the A/A genomic DNA, an oxidation current is detected only when the typing primer of SEQ. ID No. 11 is used. In contrast, as for the G/G genomic DNA, an oxidation current is detected only when the typing primer of SEQ. ID No. 12 is used. As for the A/G genomic DNA, although an oxidation current is detected when either of the typing primers of SEQ. ID NO. 11 and 12 is used, values of the oxidation currents are apparently lower than that in the above two cases.

Accordingly, in the present example, the above-described typing primers were used to provide a PCR reaction, and pyrophosphoric acid in a resultant reaction solution was analyzed using three types of enzymes, i.e., pyrophosphatase, glyceraldehyde-3-phosphate dehydrogenase, and diaphorase, thereby typing the SNP site targeted for analysis.

As described above, regarding the A/G genomic DNA, the current value measured with use of either of the typing primers of SEQ. ID Nos. 11 and 12 is apparently lower than the current value measured with use of the typing primer of SEQ. No. 11 for the A/A genomic DNA and the current value measured with use of the typing primer of SEQ. No. 12 for the G/G genomic DNA. The reason for this is that in each of the A/A genomic DNA and G/G genomic DNA, the amount of genomic DNA having A or G as a base at the SNP site which is used as a template in a PCR reaction is twice as much as that in the A/G genomic DNA.

Therefore, by conducting a quantitative analysis of current values, it is made possible to achieve SNP typing even when only one of the typing primers of SEQ. ID Nos. 11 and 12 is used. Such a quantitative analysis-based determination as to whether the SNP pattern is homo or hetero can be advantageously implemented only by the present invention which provides a clear distinction between a case where the primer extension reaction progresses and a case where the primer extension reaction does not progress.

### EXAMPLE 7

In Example 7, a region including the same SNP site as that targeted for analysis in Example 1 was amplified by a PCR reaction using two types of primers identified by SEQ. ID Nos. 13 and 21, and thereafter SNP typing was attempted using the typing primers identified by SEQ. ID Nos. 11 and 12 and the reverse primer identified by SEQ. ID No. 13.

As in Example 1, firstly, GEN [symbol] ™ (for use with blood) (Takara Shuzo Co. Ltd.) was used to extract the “A/A genomic DNA, the A/G genomic DNA and the G/G genomic DNA from bloods of three subjects whose SNP patterns are previously known to be A/A, A/G, and G/G, respectively.

Next, the primers identified by SEQ. ID Nos. 13 and 21 were used to prepare PCR reaction liquids 13 through 15, and a PCR reaction was carried out. Compositions of each PCR reaction liquid are as shown in Tables 20 through 22 below. Conditions of the PCR reaction are as shown in FIG. 11. Here, the number of repetitions is 40.

Then, the typing primers identified by SEQ. ID Nos. 11 and 12 and the reverse primer identified by SEQ. ID No. 13 were used to prepare PCR reaction liquids 16 through 21 from 1000-fold dilutions of the PCR reaction liquids 13 through 15 subjected to a PCR reaction, and a PCR reaction was carried out in each of the PCR reaction liquids 16 through 21. Compositions of each PCR reaction liquid is as shown in Tables 23 through 28 below. Conditions of the PCR reaction is as shown in FIG. 11. Here, the number of repetitions is 20.

TABLE 20
PCR reaction liquid 13

| Contents (concentration) | Volume |
|---|---|
| A/A genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Primer of SEQ. ID No. 21 (20 μM) | 0.9 μl |
| Reverse primer of SEQ. ID No. 13 (20 μM) | 0.9 μl |
| Distilled water | 13.5 μl |

TABLE 21
PCR reaction liquid 14

| Contents (concentration) | Volume |
|---|---|
| A/G genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Primer of SEQ. ID No. 21 (20 μM) | 0.9 μl |
| Reverse primer of SEQ. ID No. 13 (20 μM) | 0.9 μl |
| Distilled water | 13.5 μl |

TABLE 22
PCR reaction liquid 15

| Contents (concentration) | Volume |
|---|---|
| A/A genomic DNA (100 ng/μl) | 1 μl |
| TaKaRa Taq ™ (5 U/μl) | 0.1 μl |
| 10 × PCR buffer | 2 μl |
| dNTPs (2.5 mM) | 1.6 μl |
| Primer of SEQ. ID No. 21 (20 μM) | 0.9 μl |
| Reverse primer of SEQ. ID No. 13 (20 μM) | 0.9 μl |
| Distilled water | 13.5 μl |

TABLE 23
PCR reaction liquid 16

| Contents (concentration) | Volume |
|---|---|
| 1000-fold dilution of PCR reaction liquid 13 | 1 μl |
| after PCR reaction | |
| TaKaRa Taq ™ (5 U/μl) | 0.1 μl |
| 10 × PCR buffer | 2 μl |
| dNTPs (2.5 mM) | 1.6 μl |
| Typing primer of SEQ. ID No. 11 (20 μM) | 0.9 μl |
| Reverse primer of SEQ. ID No. 13 (20 μM) | 0.9 μl |
| Distilled water | 13.5 μl |

TABLE 24
PCR reaction liquid 17

| Contents (concentration) | Volume |
|---|---|
| 1000-fold dilution of PCR reaction liquid 13 | 1 μl |
| after PCR reaction | |
| TaKaRa Taq ™ (5 U/μl) | 0.1 μl |
| 10 × PCR buffer | 2 μl |
| dNTPs (2.5 mM) | 1.6 μl |
| Typing primer of SEQ. ID No. 12 (20 μM) | 0.9 μl |
| Reverse primer of SEQ. ID No. 13 (20 μM) | 0.9 μl |
| Distilled water | 13.5 μl |

TABLE 25
PCR reaction liquid 18

| Contents (concentration) | Volume |
|---|---|
| 1000-fold dilution of PCR reaction liquid 14 | 1 μl |
| after PCR reaction | |
| TaKaRa Taq ™ (5 U/μl) | 0.1 μl |
| 10 × PCR buffer | 2 μl |
| dNTPs (2.5 mM) | 1.6 μl |
| Typing primer of SEQ. ID No. 11 (20 μM) | 0.9 μl |
| Reverse primer of SEQ. ID No. 13 (20 μM) | 0.9 μl |
| Distilled water | 13.5 μl |

TABLE 26
PCR reaction liquid 19

| Contents (concentration) | Volume |
|---|---|
| 1000-fold dilution of PCR reaction liquid 14 | 1 μl |
| after PCR reaction | |
| TaKaRa Taq ™ (5 U/μl) | 0.1 μl |
| 10 × PCR buffer | 2 μl |
| dNTPs (2.5 mM) | 1.6 μl |
| Typing primer of SEQ. ID No. 12 (20 μM) | 0.9 μl |
| Reverse primer of SEQ. ID No. 13 (20 μM) | 0.9 μl |
| Distilled water | 13.5 μl |

TABLE 27
PCR reaction liquid 20

| Contents (concentration) | Volume |
|---|---|
| 1000-fold dilution of PCR reaction liquid 15 | 1 μl |
| after PCR reaction | |
| TaKaRa Taq ™ (5 U/μl) | 0.1 μl |
| 10 × PCR buffer | 2 μl |
| dNTPs (2.5 mM) | 1.6 μl |
| Typing primer of SEQ. ID No. 11 (20 μM) | 0.9 μl |
| Reverse primer of SEQ. ID No. 13 (20 μM) | 0.9 μl |
| Distilled water | 13.5 μl |

TABLE 28
PCR reaction liquid 21

| Contents (concentration) | Volume |
|---|---|
| 1000-fold dilution of PCR reaction liquid 15 | 1 μl |
| after PCR reaction | |
| TaKaRa Taq ™ (5 U/μl) | 0.1 μl |
| 10 × PCR buffer | 2 μl |
| dNTPs (2.5 mM) | 1.6 μl |
| Typing primer of SEQ. ID No. 12 (20 μM) | 0.9 μl |
| Reverse primer of SEQ. ID No. 13 (20 μM) | 0.9 μl |
| Distilled water | 13.5 μl |

After the PCR reaction, electrophoresis was carried out using a 3% agarose gel.

Amplification of a target DNA fragment was observed in the PCR reaction liquids 16, 18, 19, and 21. On the other hand, almost no amplification was observed in the PCR reaction liquids 17 and 20. That is, regarding the A/A genomic DNA, the amplification was observed only when the typing primer of SEQ. ID No. 11 was used. In contrast, as for the G/G genomic DNA, the amplification was observed only when the typing primer of SEQ. ID No. 12 was used. As for the A/G genomic DNA, the amplification was observed when either of the typing primers was used.

Consequently, it was confirmed that a base in the SNP site can be determined using the typing primers of SEQ. ID Nos. 11 and 12.

Regarding the amount of amplification of the DNA fragment, substantially the same amount was observed in the PCR reaction liquids 16 and 21, and the amount observed in each of the PCR reaction liquids 18 and 19 was apparently lower than the amount observed in each of the PCR reaction liquids 16 and 21. In this manner, there is confirmed a clear difference in the amount of amplification of the DNA fragment between the reaction liquids 16 and 18, although the same typing primer was used. This is because as compared to the PCR reaction liquid 18, the PCR reaction liquid 16 has twice the amount of genomic DNA having A as a base at the SNP site which is used as a template in a PCR reaction.

The above results show that it is also possible to analyze whether the SNP pattern of a genomic DNA is homo or hetero based on a difference in the amount of amplification of the DNA fragment. Accordingly, in the present example, even if only either one of the typing primers of SEQ. ID Nos. 11 and 12 is used, it is possible to type three kinds of SNP patterns of A/A, A/G and G/G by analyzing not only the presence or absence of amplification of the DNA fragment but also the amount of amplification of the DNA fragment. Such a quantitative SNP pattern analysis based on the amount of amplification of the DNA fragment is made possible only by the typing primer of the present invention which provides a clear distinction between a case where the primer extension reaction progresses and a case where the primer extension reaction does not progress.

The above results show that SNP typing can be achieved by using the typing primer of the present invention.

A base type determination method of the present invention provides accurate and reproducible determination of the type of a target base in a nucleic acid, and therefore is advantageously used with a medical device or in the filed of pharmaceuticals.

While the invention has been described in detail, the foregoing description is in all aspects illustrative and not restrictive. It is understood that numerous other modifications and variations can be devised without departing from the scope of the invention.

What is claimed is:

1. A base type determination method for determining a base type of a monobasic substituted region of a target nucleic acid, the method comprising the steps of:
(a) preparing a solution containing a target double-stranded nucleic acid having the monobasic substituted region, a base type determination primer, a DNA polymerase, and dNTPs;
(b) causing the base type determination primer to hybridize to the target double-stranded nucleic acid in the solution, and causing a primer extension reaction to start progressing from the base type determination primer; and
(c) analyzing the degree of progress of the primer extension reaction to determine the base type of the substituted region,
wherein the base type determination primer consists of a first single-stranded nucleic acid which is capable of, when hybridizing to the target double-stranded nucleic acid, hybridizing to one of two strands of the target double-stranded nucleic acid such that a 3′ terminal of the primer corresponds to the substituted region of said one strand of the target double-stranded nucleic acid, and
wherein the first single-stranded nucleic acid consists of:
a substitution corresponding region which is located at the 3′ terminal and consists of one base complementary to any one of predictable types of bases in the substituted region of said one strand of the target double-stranded nucleic acid;
an uncomplementary region which is located adjacent to the substitution corresponding region on a 5′ terminal side thereof and consists of two bases uncomplementary to said one strand of the target double-stranded nucleic acid; and
a complementary region which is located adjacent to the uncomplementary region on the 5′ terminal side thereof and consists of five or more bases complementary to said one strand of the target double-stranded nucleic acid.

2. The base type determination method according to claim 1, wherein the DNA polymerase has substantially no 3′→5′ exonuclease activity.

3. The base type determination method according to claim 1, wherein the first single-stranded nucleic acid is a DNA.

4. The base type determination method according to claim 1,
wherein in the step (a), the solution further contains a reverse primer consisting of a single-stranded nucleic acid capable of hybridizing to the other strand of the target double-stranded nucleic acid, and
wherein in the step (b), the primer extension reaction is caused to progress using a base sequence amplifying method selected from the group consisting of a PCR, an SDA, an RCR, an LAMP, and a TMA.

5. The base type determination method according to claim 4, wherein in the step (c), the base type of the substituted region is determined based on a difference in progress of the primer extension reaction.

6. The base type determination method according to claim 4, wherein in the step (c), the degree of progress of the primer extension reaction is analyzed by using a method selected from the group consisting of electrophoresis, mass analysis, and liquid chromatography to measure the amount of amplification of a base sequence amplified by the base sequence amplifying method.

7. The base type determination method according to claim 1, wherein in the step (c), the degree of progress of the primer extension reaction is analyzed by measuring the amount of pyrophosphoric acid generated by the primer extension reaction.

8. The base type determination method according to claim 4, wherein in the step (c), the amount of amplification of a base sequence amplified by the base sequence amplifying method is measured by measuring the amount of pyrophosphoric acid generated by the primer extension reaction.

9. The base type determination method according to claim 4, wherein in the step (c), the amount of amplification of a base sequence amplified by the base sequence amplifying method is quantitatively analyzed to determine the base type of the substituted region.

10. The base type determination method according to claim 7,
wherein measurement of the amount of pyrophosphoric acid includes the steps of:
converting the pyrophosphoric acid into an inorganic phosphoric acid within a sample containing at least a portion of the solution resulted from the step (b);
providing the sample to a measurement system including glyceraldehyde 3-phosphate, oxidized nicotinamide adenine dinucleotide, glyceraldehyde 3-phosphatedehyrogenase, and at least one electron-transfer mediator; and
measuring a value of current generated in the measurement system, and
wherein the value of current indicates a concentration of the pyrophosphoric acid in the sample.

11. The base type determination method according to claim 10, wherein said at least one electron-transfer mediator is selected from the group consisting of ferricyanide, 1,2-naphthoquinone-4-sulfonic acid, 2,6-dichlorophenol-indophenol, dimethylbenzoquinone, 1-methoxy-5-methylphenazinium sulfate, methylene blue, gallocyanine, thionine, phenazine methosulfate, and meldora blue.

12. The base type determination method according to claim 10, wherein the measurement system further include diaphorase.

13. The base type determination method according to claim 10, wherein the pyrophosphoric acid is converted into the inorganic phosphoric acid by causing the pyrophosphoric acid to react with pyrophosphatase in the sample.

14. The base type determination method according to claim 7,
wherein measurement of the amount of the pyrophosphoric acid includes the steps of:
placing a sample including at least a portion of a solution resulted from the step (b) in one region of a measurement system having at least two regions divided by a membrane which holds H⁺-pyrophosphatase and has a limited permeability to H⁺; and
measuring a change in concentration of H⁺ in either one of said at least two regions of the measurement system, and
wherein the degree of the change in concentration of H⁺ indicates the concentration of the pyrophosphoric acid in the sample.

15. The base type determination method according to claim 14,
wherein the measurement of the pyrophosphoric acid includes the steps of:
providing the sample including at least a portion of a solution resulted from the step (b) to a measurement system including an artificial or natural membrane vesicle containing H⁺-pyrophosphatase therein; and
measuring the change in concentration of H⁺ in the inside or outside of the membrane vesicle, and
wherein the degree of the change in concentration of H⁺ indicates the concentration of the pyrophosphoric acid in the sample.

16. The base type determination method according to claim 14, wherein the change in concentration of H⁺ is measured by either a method which measures an optical change converted from the change in concentration of H⁺ or a method which measures an electrical change converted from the change in concentration of H⁺.

17. The base type determination method according to claim 16, wherein the method which measures an optical change uses a pH test paper, a pH-sensitive dye, or a membrane potential-sensitive dye.

18. The base type determination method according to claim 16, wherein the method which measures an electrical change is selected from the group consisting of a metal electrode method, a glass electrode method, an ISFET electrode method, a patch-clamp method, and an LAPS method.

19. The base type determination method according to claim 17, wherein the method which measures an optical change uses the pH-sensitive dye to measure the change in concentration of H⁺ in the inside of the membrane vesicle.

20. The base type determination method according to claim 1,
wherein in the step (a), the solution further contains a second base type determination primer,
wherein the second base type determination primer consists of a second single-stranded nucleic acid capable of, when hybridizing to the target double-stranded nucleic acid, hybridizing to one of two strand of the target double-stranded nucleic acid which is the same strand as that to which the first base type determination primer is supposed to hybridize, such that a 3′ terminal of the second base type determination primer corresponds to the substituted region of said one strand, and
wherein the second single-stranded nucleic acid includes:
a second substitution corresponding region located at the 3′ terminal and consisting of one base which is complementary to any one of predictable types of bases in the substituted region of the target double-stranded nucleic acid and is different in type from said one base of the substitution corresponding region of the first single-stranded nucleic acid;
a second uncomplementary region which is adjacent to the second substitution corresponding region on the 5′ terminal side and consists of two bases uncomplementary to said one strand of the target double-stranded nucleic acid; and
a second complementary region which is adjacent to the second uncomplementary region on the 5′ terminal side and consists of five or more bases complementary to said one strand of the target double-stranded nucleic acid.

21. The base type determination method according to claim 20, wherein the first single-stranded nucleic acid and the second single-stranded nucleic acid are different in length from each other.

22. The base type determination method according to claim 20, wherein the first single-stranded nucleic acid and the second single-stranded nucleic acid are labeled by their respective fluorescences which are different in wavelength.

23. A base type determination primer for determining a base type of a monobasic substituted region of a target nucleic acid,
wherein the primer consists of a single-stranded nucleic acid which is capable of hybridizing to the target nucleic acid such that a 3′ terminal of the primer corresponds to the substituted region of the target nucleic acid, and
wherein the single-stranded nucleic acid includes:
a substitution corresponding region which is located at the 3′ terminal and consists of one base complementary to any one of predictable types of bases in the substituted region of the target nucleic acid;
an uncomplementary region which is located adjacent to the substitution corresponding region on a 5′ terminal side thereof and consists of two bases uncomplementary to the target nucleic acid; and
a complementary region which is located adjacent to the uncomplementary region on the 5′ terminal side thereof and consists of five or more bases complementary to the target nucleic acid.

24. A base type determination reagent kit for determining a base type of a monobasic substituted region of a target nucleic acid, the kit comprising a base type determination primer, a DNA polymerase, and dNTPs,
wherein the primer consists of a first single-stranded nucleic acid which is capable of hybridizing to the target nucleic acid such that a 3′ terminal of the primer corresponds to the substituted region of the target nucleic acid, and
wherein the first single-stranded nucleic acid includes:
a substitution corresponding region which is located at the 3′ terminal and consists of one base complementary to any one of predictable types of bases in the substituted region of the target nucleic acid;
an uncomplementary region which is located adjacent to the substitution corresponding region on a 5′ terminal side thereof and consists of two bases uncomplementary to the target nucleic acid; and
a complementary region which is located adjacent to the uncomplementary region on the 5′ terminal side thereof and consists of five or more bases complementary to the target nucleic acid.

25. The base type determination reagent kit according to claim 24, wherein the DNA polymerase has substantially no 3′→5′ exonuclease activity.

26. The base type determination reagent kit according to claim 24, wherein the first single-stranded nucleic acid is a DNA.

27. The base type determination reagent kit according to claim 24, further comprising a reverse primer.

28. The base type determination reagent kit according to claim 24, further comprising pyrophosphatase.

29. The base type determination reagent kit according to claim 28, further comprising glyceraldehyde 3-phosphate, oxidized nicotinamide adenine dinucleotide, glyceraldehyde 3-phosphatedehyrogenase, and at least one electron-transfer mediator.

30. The base type determination reagent kit according to claim 29, further comprising diaphorase.

31. The base type determination reagent kit according to claim 29, wherein said at least one electron-transfer mediator is selected from the group consisting of ferricyanide, 1,2-naphthoquinone-4-sulfonic acid, 2,6-dichlorophenol-indophenol, dimethylbenzoquinone, 1-methoxy-5-methylphenazinium sulfate, methylene blue, gallocyanine, thionine, phenazine methosulfate, and meldora blue.

32. The base type determination reagent kit according to claim 24, further comprising H⁺-pyrophosphatase.

33. The base type determination reagent kit according to claim 32, further comprising a pH test paper, a pH-sensitive dye, or a membrane potential-sensitive dye.

34. The base type determination reagent kit according to claim 24, further comprising a second base type determination primer,
wherein the second base type determination primer consists of a second single-stranded nucleic acid capable of hybridizing to the target nucleic acid such that the 3′ terminal corresponds to the substituted region of the same strand as that to which the first base type determination primer is supposed to hybridize, and
wherein the second single-stranded nucleic acid includes:
a second substitution corresponding region located at the 3′ terminal and consisting of one base which is complementary to any one of predictable types of bases in the substituted region of the target nucleic acid and is different in type from said one base of the substitution corresponding region of the first single-stranded nucleic acid;
a second uncomplementary region which is located adjacent to the second substitution corresponding region on a 5′ terminal side thereof and consists of two bases uncomplementary to the target nucleic acid; and
a second complementary region which is located adjacent to the second uncomplementary region on the 5′ terminal side thereof and consists of five or more bases complementary to the target nucleic acid.

35. The base type determination reagent kit according to claim 34, wherein the first single-stranded nucleic acid and the second single-stranded nucleic acid are different in length from each other.

36. The base type determination reagent kit according to claim 34, wherein the first single-stranded nucleic acid and the second single-stranded nucleic acid are labeled by their respective fluorescences which are different in wavelength.
